(19) United States
(12) Patent Application Publication
(10) Pub. No.: US 2020/0303078 A1
(43) Pub. Date: Sep. 24, 2020

(54) Systems and Methods for Deriving and Optimizing Classifiers from Multiple Datasets

(71) Applicant: Inflammatix, Inc., Burlingame, CA (US)

(72) Inventors: Michael B. Mayhew, Burlingame, CA (US); Ljubomir Buturovic, Burlingame, CA (US); Timothy E. Sweeney, Burlingame, CA (US); Roland Luethy, Burlingame, CA (US); Purvesh Khatri, Burlingame, CA (US)

(73) Assignee: Inflammatix, Inc., Burlingame, CA (US)

(21) Appl. No.: 16/826,042

(22) Filed: Mar. 20, 2020

Related U.S. Application Data
(60) Provisional application No. 62/822,730, filed on Mar. 22, 2019.

(51) Int. Cl. G16H 50/30; G16B 20/00; G16B 40/00; G06N 7/00; G06N 3/08; G06N 20/10; G06N 20/20

(52) U.S. Cl. CPC G16H 50/30; G16B 20/00; G16B 40/00; G06N 20/20; G06N 3/08; G06N 20/10; G06N 7/005

(57) ABSTRACT

Systems and methods for subject clinical condition evaluation using a plurality of modules are provided. Modules comprise features whose corresponding feature values associate with an absence, presence or stage of phenotypes associated with the clinical condition. A first dataset is obtained having feature values, acquired through a first technical background from respective subjects in transcriptomic, proteomic, or metabolomic form, for at least a first of the plurality of modules. A second training dataset is obtained having feature values, acquired through a technical background other than the first technical background, from training subjects of the second dataset, in the same form as for the first dataset, of at least the first module. Inter-dataset batch effects are removed by co-normalizing feature values across the training datasets, thereby calculating co-normalized feature values used to train a classifier for clinical condition evaluation of the test subject.

### CROSS REFERENCE TO RELATED APPLICATION

This application claims priority to U.S. Provisional Patent Application No. 62/822,730, filed Mar. 22, 2019, the content of which is hereby incorporated by reference in its entirety for all purposes.

### TECHNICAL FIELD

This disclosure relates to the training and implementation of machine learning classifiers for the evaluation of the clinical condition of a subject.

### BACKGROUND

Biological modeling methods that rely on transcriptomics and/or other ‘omic’-based data, e.g., genomics, proteomics, metabolomics, lipidomics, glycomics, etc., can be used to provide meaningful and actionable diagnostics and prognostics for a medical condition. For example, several commercial genomic diagnostic tests are used to guide cancer treatment decisions. The Oncotype IQ suite of tests (Genomic Health) are examples of such genomic-based assays that provide diagnostic information guiding treatment of various cancers. For instance, one of these tests, ONCOTYPE DX® for breast cancer (Genomic Health) queries 21 genomic alleles in a patient's tumor to provide diagnostic information guiding treatment of early-stage invasive breast cancers, e.g., by providing a prognosis for the likely benefit of chemotherapy and the likelihood or recurrence. See, for example, Paik et al., 2004, N Engl J Med. 351, pp. 2817-2825 and Paik et al., 2016, J Clin Oncol. 24(23), pp. 3726-3734.

High-throughput ‘omics’ technologies, such as gene expression microarrays, are often used to discover smaller targeted biomarker panels. However, such datasets always have more variables than samples, and so are prone to non-reproducible, overfit results. See., for example, Shi et al., 2008, BMC Bioinformatics, 9(9), p. S10 and Ioannidis et al., 2001, Nat Genet. 29(3), pp. 306-09. Moreover, in an effort to increase statistical power, biomarker discovery is usually performed in a clinically homogeneous cohort using a single type of assay, e.g., a single type of microarray. Although this homogeneous design does result in a greater statistical power, the results are less likely to remain true in different clinical cohorts using different laboratory techniques. As a result, multiple independent validations are necessary for any new classifier derived from high-throughput studies.

Fortunately, technological advances have resulted in the development of many different types of high-throughput biological data assays. This, in turn, has led to performance of large clinical studies on the biological effects of many different medical disorders. Vast collections of omics-based datasets are found on-line, for example, in the Gene Expression Omnibus (GEO) hosted by the National Center for Biotechnology Information (NCBI) and the ArrayExpress Archive of Functional Genomic hosted by the European Bioinformatics Institute (EMBL-EBI). These and other datasets, many of which are publically available, are a good source for training machine learning classifiers to distinguish, for example, between various disease states and expected treatment outcomes, particularly because they utilize different clinical cohorts and different laboratory techniques. In theory, better classifiers could be trained using these diverse datasets, because assay-specific and batch-specific effects of individual patient cohorts and assay techniques can be identified and ignored, while emphasizing the phenotypic effects caused by the underlying biology.

However, classifier training against heterogeneous datasets, e.g., that are collected from multiple studies and/or using multiple assay platforms, is problematic because feature values, e.g., expression levels, are not comparable across the different studies and assay platforms. That is, the inclusion of multiple datasets from different technical and biological backgrounds leads to substantial heterogeneity between included datasets. If not removed, such heterogeneity can confound the construction of a classifier across datasets. Conventional approaches for training a classifier using heterogeneous datasets simply optimize a parameterized classifier in a single cohort, and then apply it externally. However, the different technical backgrounds preclude direct application in external datasets, and so classifiers are often retrained locally, leading to strongly biased estimates of performance. See, Tsalik et al, 2016; and Sci Transl Med 8, 322ra311. In another approach, non-parameterized classifiers are optimized across multiple datasets that had not been co-normalized, as there was no way to also optimize these classifiers in a pooled setting. See Sweeney et al, 2015, Sci Transl Med 7(287), pp. 287ra71; and Sweeney et al., 2016, Sci Transl Med 8(346), pp. 346ra91. Finally, in recently published work, a group from Sage Bionetworks attempted to learn parameterized models across multiple pooled datasets that were NOT properly co-normalized. However, as reported, these model performed poorly in validation. See, Sweeney et al., 2018, Nature Communications 9, 694.

### SUMMARY

In view of the background above, improved methods and systems for developing and implementing more robust and generalizable machine learning classifiers are needed in the art. Advantageously, the present disclosure provides technical solutions (e.g., computing systems, methods, and non-transitory computer readable storage mediums) addressing these and other problems in the field of medical diagnostics. For instance, in some embodiments, the present disclosure provides methods and systems that use heterogeneous repositories of input molecular (e.g. genomic, transcriptomic, proteomic, metabolomics) and/or clinical data with associated clinical phenotypes to generate machine learning classifiers, e.g., for diagnosis, prognosis, or clinical predictions, that are more robust and generalizable than conventional classifiers.

Significantly, as described herein, non-conventional co-normalization techniques have been developed that reduce the impact of dataset differences and bring the data into a single pooled format. Appropriately co-normalized heterogeneous datasets unlock the potential of machine learning by integrating and overcoming clinical heterogeneity to produce generalizable, accurate classifiers. Accordingly, the methods and systems described herein allow for a breakthrough in development of novel classifiers using multiple datasets.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

In some embodiments, the present disclosure provides methods and systems for implementing those methods for training a neural network classifier based on heterogeneous repositories of input molecular (e.g. genomic, transcriptomic, proteomic, metabolomics) and clinical data with associated clinical phenotypes. In some embodiments, the method includes identifying biomarkers, a priori, that have statistically significant differential feature values (e.g., gene expression values) in a clinical condition of interest, and determining the sign or direction of each biomarker's feature value(s) in the clinical condition, e.g., positive or negative. In some embodiments, multiple datasets are collected that generally examine the same clinical condition, e.g., a medical condition such as the presence of an acute infection. The raw data from each of these datasets is then normalized using a study-specific procedure, e.g., using a robust multi-array average (RMA) algorithm to normalize gene expression microarray data or Bowtie and Tophat algorithms to normalize RNA sequencing (RNA-Seq) data. The normalized data from each of these datasets is then mapped to a common variable and co-normalized with the other datasets. Finally, the co-normalized and mapped datasets are then used to construct and train a neural network classifier, in which input units corresponding to identified biomarkers with statistically significant differential feature values having shared signs of effect, e.g., positive or negative, on the clinical condition status are each grouped into ‘modules’ using uniformly-signed coefficients to preserve direction of module gene effects.

For instance, in one aspect, the present disclosure provides methods and systems for performing such methods for evaluating a clinical condition of a test subject of a species using an a priori grouping of features, where the a priori grouping of features includes a plurality of modules. Each module in the plurality of modules includes an independent plurality of features whose corresponding feature values each associate with an absence, presence, or stage of an independent phenotype associated with the clinical condition. The method includes obtaining in electronic form a first training dataset, where the first training dataset includes, for each respective training subject in a first plurality of training subjects of the species: (i) a first plurality of feature values, acquired through a first technical background using a biological sample of the respective training subject, for the independent plurality of features, in a first form that is one of transcriptomic, proteomic, or metabolomic, of at least a first module in the plurality of modules and (ii) an indication of the absence, presence or stage of a first independent phenotype corresponding to the first module, in the respective training subject. The method then includes obtaining in electronic form a second training dataset, where the second training dataset includes, for each respective training subject in a second plurality of training subjects of the species: (i) a second plurality of feature values, acquired through a second technical background other than the first technical background using a biological sample of the respective training subject, for the independent plurality of features, in a second form identical to the first form, of at least the first module and (ii) an indication of the absence, presence or stage of the first independent phenotype in the respective training subject. The method then includes co-normalizing feature values for features present in at least the first and second training datasets across at least the first and second training datasets to remove an inter-dataset batch effect, thereby calculating, for each respective training subject in the first plurality of training subjects and for each respective training subject in the second plurality of training subjects, co-normalized feature values of at least the first module for the respective training subject. The method then includes training a main classifier, against a composite training set, to evaluate the test subject for the clinical condition, the composite training set including, for each respective training subject in the first plurality of training subjects and for each respective training subject in the second plurality of training subjects: (i) a summarization of the co-normalized feature values of the first module and (ii) an indication of the absence, presence, or stage of the first independent phenotype in the respective training subject.

In another aspect, the present disclosure provides methods and systems for performing such methods for evaluating a clinical condition of a test subject of a species. The method includes obtaining in electronic form a first training dataset, where the first training dataset includes, for each respective training subject in a first plurality of training subjects of the species: (i) a first plurality of feature values, acquired using a biological sample of the respective training subject, for a plurality of features and (ii) an indication of the absence, presence or stage of a first independent phenotype in the respective training subject. The first independent phenotype represents a diseased condition, and a first subset of the first training dataset consists of subjects that are free of the diseased condition. The method then includes obtaining in electronic form a second training dataset, where the second training dataset includes, for each respective training subject in a second plurality of training subjects of the species: (i) a second plurality of feature values, acquired using a biological sample of the respective training subject, for the plurality of features and (ii) an indication of the absence, presence, or stage of the first independent phenotype in the respective training subject. A first subset of the second training dataset consists of subjects that are free of the diseased condition. The method then includes co-normalizing feature values for a subset of the plurality of features across at least the first and second training datasets to remove an inter-dataset batch effect, where the subset of features is present in at least the first and second training datasets. The co-normalizing includes estimating an inter-dataset batch effect between the first and second training dataset using only the first subset of the respective first and second training datasets. The inter-dataset batch effect includes an additive component and a multiplicative component and the co-normalizing solves an ordinary least-squares model for feature values across the first subset of the respective first and second training datasets and shrinks resulting parameters representing the additive component and a multiplicative component using an empirical Bayes estimator, thereby calculating using the resulting parameters: for each respective training subject in the first plurality of training subjects and for each respective training subject in the second plurality of training subjects, co-normalized feature values of the subset of the plurality of features. The method then includes training a main classifier, against a composite training set, to evaluate the test subject for the clinical condition, the composite training set including: for each respective training subject in the first plurality of training subjects and for each respective training subject in the second plurality of training subjects: (i) co-normalized feature values of the subset of the plurality of features and (ii) the indication of the absence, presence, or stage of the first independent phenotype in the respective training subject.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIGS. 1A and 1B collectively illustrate an example block diagram for a computing device in accordance with some embodiments of the present disclosure.

FIGS. 2A, 2B, 2C, 2D, 2E, 2F, 2G, 2H, and 2I illustrate an example flowchart of a method of classifying a subject in accordance with some embodiments of the present disclosure in which optional steps are indicated by dashed boxes.

FIG. 3 illustrates a network topology in which plurality of modules at the bottom each contribute a geometric mean of genes known a priori to all move in the same direction, on average, in the clinical condition of interest. Outputs at the top of the network are the clinical conditions of interest (bacterial infection—I_{bac}, viral infection Iᵥᵢᵣₐ, no infection—Iₙₒₙ) in accordance with some embodiments of the present disclosure.

FIG. 4 illustrates a network topology in which minispoke networks are used for each module (one of which is shown in more detail in the right portion of the figure). Individual biomarkers are summarized by a local network (instead of summarized by their geometric mean) and then passed into the main classification network.

FIGS. 5A and 5B illustrate iterative COCONUT alignment in which “reference” is microarray data, “Target” is NanoString data in accordance with an embodiment of the present disclosure. The graphs show distributions across healthy samples of NanoString gene expression and microarray gene expression, for two genes (5A—HK3, 5B—IFI27) from the set of 29. The microarray distributions are shown at three distinct iterations in the co-normalization-based alignment process. Dashed lines indicate distributions at intermediate iterations, solid lines show the distribution at termination of the procedure.

FIGS. 6A and 6B illustrate the distributions of co-normalized expression values of bacterial, viral and non-infected training set samples for selected genes (6A—fever markers) (6B—severity markers) of the set of 29 genes in a training dataset used in an example of the present disclosure.

FIGS. 7A and 7B respectively illustrate the two-dimensional (7A) and three-dimensional (7B) t-SNE projection of the co-normalized expression values of the 29 genes across the training dataset in which each subject is labeled bacterial, viral, or non-infected in accordance with an embodiment of the present disclosure.

FIGS. 8A and 8B respectively illustrate the two-dimensional (8A) and three-dimensional (8B) principal component analysis plot of the co-normalized expression values of the 29 genes across the training dataset in which each subject is labeled bacterial, viral, or non-infected in accordance with an embodiment of the present disclosure.

FIG. 9 illustrates the two-dimensional principal component analysis plot of the co-normalized expression values of the 29 genes across the training dataset in which each subject is labeled by source study in accordance with an embodiment of the present disclosure.

FIGS. 10A, 10B, 10C, 10D, 10E, and 10F and FIGS. 10G, 10H, 10I, 10J, 10K, and 10L respectively illustrates analysis of validation performance bias using 6 geometric mean scores instead of direct expression values of the 29 genes in accordance with an embodiment of the present disclosure in which FIGS. 10A, 10B, and 10C are logistic regression, FIGS. 10D, 10E, and 10F are XGBoost, FIGS. 10G, 10H, and 10I are support vector machine with the RBF kernel, and FIGS. 10J, 10K, and 10L are multi-layer perceptrons. The x-axis is the difference between outer fold and inner fold average pairwise area-under-the-ROC (APA) curve for the top 10 models, as ranked by cross validation APA, of each model type. Each dot corresponds to a model. The y-axis corresponds to the outer fold APA. The vertical dashed line indicates no difference between APA in the inner loop and outer loop.

FIGS. 11A, 11B, 11C, 11D, 11E, and 11F and FIGS. 11G, 11H, 11I, 11J, 11K, and 11L respectively illustrates analysis of validation performance bias using direct expression values of the 29 genes in accordance with an embodiment of the present disclosure in which FIGS. 11A, 11B, and 11C are logistic regression, FIGS. 11D, 11E, and 11F are XGBoost, FIGS. 11G, 11H, and 11I are support vector machine with the RBF kernel, and FIGS. 11J, 11K, and 11L are multi-layer perceptrons. The x-axis is the difference between outer fold and inner fold average pairwise area-under-the-ROC (APA) curve for the top 10 models, as ranked by cross validation APA, of each model type. Each dot corresponds to a model. The y-axis corresponds to the outer fold APA. The vertical dashed line indicates no difference between APA in the inner loop and outer loop.

FIG. 12 illustrates pseudocode for iterative application of the COCONUT algorithm, in accordance with some embodiments of the present disclosure.

FIG. 13 illustrates an example flowchart of a method for training a classifier to evaluate a clinical condition of a subject, in accordance with some embodiments of the present disclosure.

FIG. 14 illustrates an example flowchart of a method of evaluating a clinical condition of a subject, in accordance with some embodiments of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for generating and using machine learning classifiers for diagnosing, providing a prognosis, or providing a clinical prediction for a medical condition. In particular, the methods and systems provided herein facilitate the use of heterogeneous repositories of molecular (e.g. genomic, transcriptomic, proteomic, metabolomic) and/or clinical data with associated clinical phenotypes for training machine learning classifiers with improved performance.

In some embodiments, as described herein, the disclosed methods and systems achieve machine learning classifiers with improved performance by estimating an inter-dataset batch effect between heterogenous training datasets.

In some embodiments, the systems and methods described herein leverage co-normalization methods developed to bring multiple discrete datasets into a single pooled data framework. These methods improve classifier performance on the overall pooled accuracy, some averaging function of individual dataset accuracy within the pooled framework, or both. Those skilled in the art will recognize that this ability requires improved co-normalization of heterogeneous datasets, which is not a feature of traditional omics-based data science pipelines.

In some embodiments, an initial step in the classifier training methods described herein is a priori identification of biomarkers to train against. Biomarkers of interest can be identified using a literature search, or within a ‘discovery’ dataset in which a statistical test is used to select biomarkers that are associated with the clinical condition of interest. In some embodiments, the biomarkers of interest are then grouped according to the sign of their direction of change in the clinical decision of interest.

In some embodiments, subsets of variables for training these classifiers are selected from known molecular variables (e.g., genomic, transcriptomic, proteomic, metabolomic data) present in the heterogeneous datasets. In some embodiments, these variables are selected using statistical thresholding for differential expression using tools such as Significance Analysis for Microarrays (SAM), or meta-analysis between datasets, or correlations with class, or other methods. In some embodiments, the available data is expanded by engineering new features based on the patterns of molecular profiles. These new features may be discovered using unsupervised analyses such as denoising autoencoders, or supervised methods such as pathway analysis using existing ontologies or pathway databases (such as KEGG).

In some embodiments, datasets for training the classifier are obtained from public or private sources. In the public domain, repositories such as NBCI GEO or ArrayExpress (if using transcriptomic data) can be utilized. The datasets must have at least one of the classes of interest present, and, if using a co-normalization function that requires healthy controls, they must have healthy controls. In some embodiments, only data of a single biologic type is gathered (e.g., only transcriptomic data, but not proteomic data), but may be from widely different technical backgrounds (e.g. both RNAseq and DNA microarrays).

In some embodiments, input data is stratified to ensure that approximately equal proportions of each class are present in each input dataset. This step avoids confounding by the source of heterogeneous data in learning a single classifier across pooled datasets. Stratification may be done once, multiple times, or not at all.

In some embodiments, when raw data from the original technical format is obtained, standardized within-datasets normalization procedures are performed, in order to minimize the effect of varying normalization methods on the final classifier. Data from technical platforms of the same type are preferably normalized in the same manner, typically using general procedures such as background correction, log² transformation, and quantile normalization. Platform-specific normalization procedures are also common (e.g. gcRMA for Affymetrix platforms with positive-match controls). The result is a single file or other data structure per dataset.

In some embodiments, co-normalization is then performed in two steps, optional inter-platform common variable mapping followed by necessary co-normalization.

Inter-platform common variable mapping is necessary in those instances where the platforms drawn upon for the datasets do not follow the same naming conventions and/or measure the same target with multiple variations (e.g., many RNA microarrays have degenerate probes for single genes). A common reference (e.g., mapping to RefSeq genes) is chosen, and variables are relabeled (in the single case) or summarized (in the multiple-variable case; e.g. by taking a measure of central tendency such as median, mean, etc., or fixed-effect meta-analysis of degenerate probes for the same gene).

Co-normalization is necessary because, having identified variables with common names between datasets, it is often the case that those variables have substantially different distributions between datasets. These values, thus, are transformed to match the same distributions (e.g., mean and variance) between datasets. The co-normalization can be performed using a variety of methods, such as COCONUT (Sweeney et al., 2016, Sci Transl Med 8(346), pp. 346ra91; and Abouelhoda et al., 2008, BMC Bioinformatics 9, p. 476), quantile normalization, ComBat, pooled RMA, pooled gcRMA, or invariant-gene (e.g., housekeeping) normalization, among others.

In some embodiments, data that is co-normalized using the improved methods described herein is subjected to machine learning, to train a main classifier for the classes of a clinical condition of interest, e.g., disease diagnostic or prognostic classes. In non-limiting examples, this may make use of linear regression, penalized linear regression, support vector machines, tree-based methods such as random forests or decision trees, ensemble methods such as adaboost, XGboost, or other ensembles of weak or strong classifiers, neural net methods such as multi-layer perceptrons, or other methods or variants thereof. In some embodiments, the main classifier may learn directly from the selected variables, from engineered features, or both. In some embodiments, main classifier is an ensemble of classifiers.

In some embodiments, these methods and systems are further augmented by generating new samples from the pooled data by means of a generative function. In some embodiments, this includes adding random noise to each sample. In some embodiments, this includes more complex generative models such as Boltzmann machines, deep belief networks, generative adverse networks, adversarial autoencoders, other methods, or variants thereof.

In some embodiments, the methods and systems for classifier development include cross-validation, model selection, model assessment, and calibration. Initial cross-validation estimates performance of a fixed classifier. Model selection uses hyperparameter search and cross-validation to identify the most accurate classifier. Model assessment is used to estimate performance of the selected model in independent data, and can be performed using leave-one-dataset-out (LODO) cross validation, nested cross-validation, or bootstrap-corrected performance estimation, among others. Calibration adjusts classifier scores to distribution of phenotypes observed in clinical practice, for the purpose of converting the scores to intuitive, human-interpretable values. It can be performed using methods such as the Hosmer-Lemeshow test and calibration slope.

In some embodiments, a neural-net classifier such as a multilayer perceptron is used for supervised classification of an outcome of interest (such as the presence of an infection) in the co-normalized data. The variables that are known to move together on average in the clinical condition of interest are grouped into ‘modules’, and a neural network architecture that interprets these grouped modules is learned above.

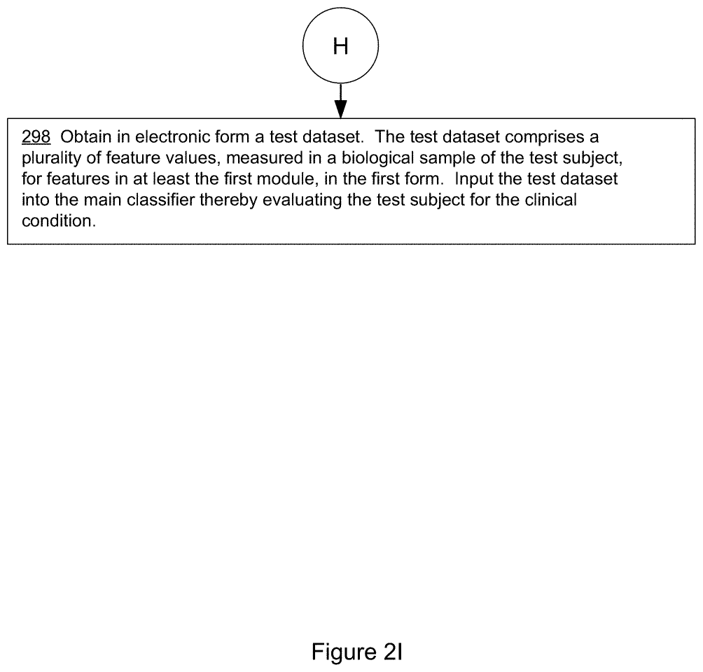

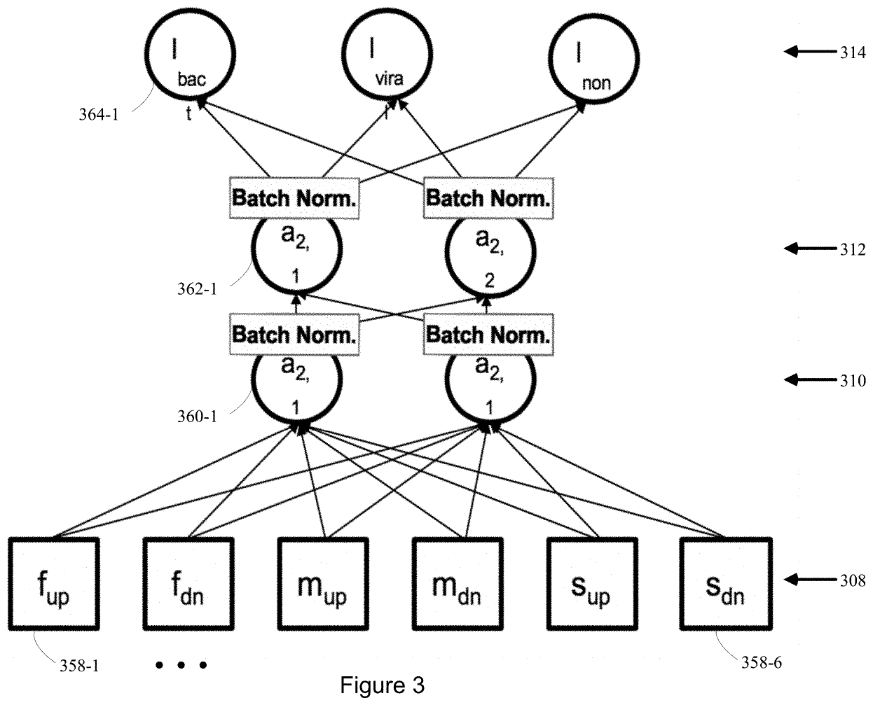

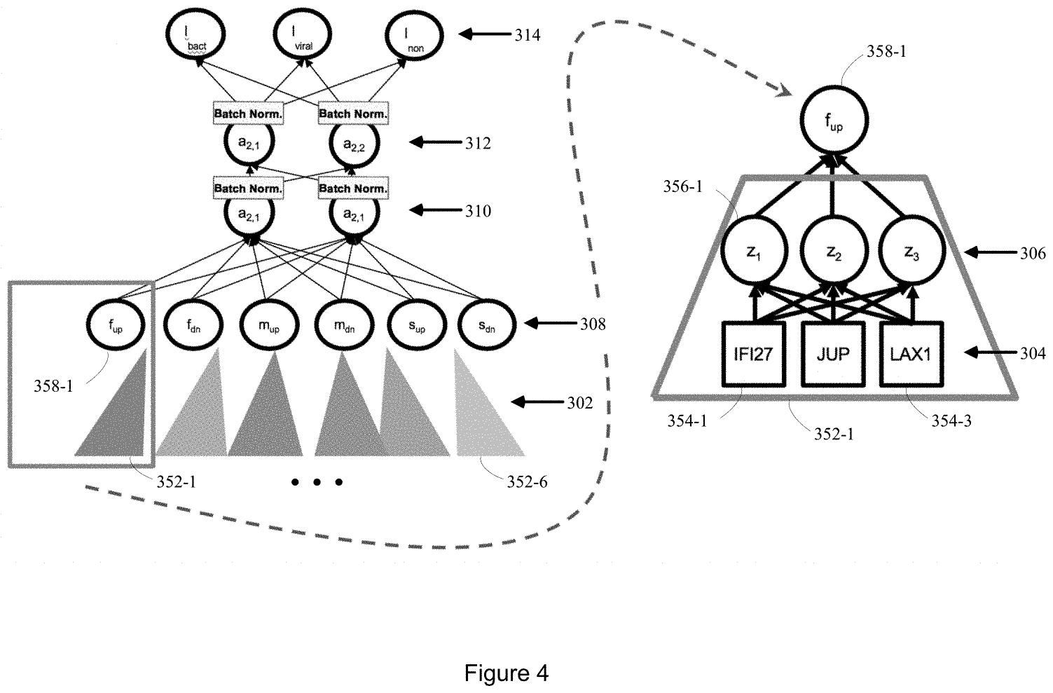

In some embodiments, the ‘modules’ are constructed in one of two ways. In the first way, the biomarkers within the module are grouped by taking a measure of their central tendency, such as geometric mean, and feeding this into a main classifier (e.g., as illustrated in FIG. 3). In another embodiment, a ‘spoke’ network is constructed, where the inputs are the biomarkers in the module, and they are interpreted via a component classifier that feeds into the main classifier (e.g., as illustrated in FIG. 4).

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As disclosed herein, the terms “nucleic acid” and “nucleic acid molecule” are used interchangeably. The terms refer to nucleic acids of any composition form, such as deoxyribonucleic acid (DNA), ribonucleic acid (RNA), and/or DNA or RNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As disclosed herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject.

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

### Exemplary System Embodiments

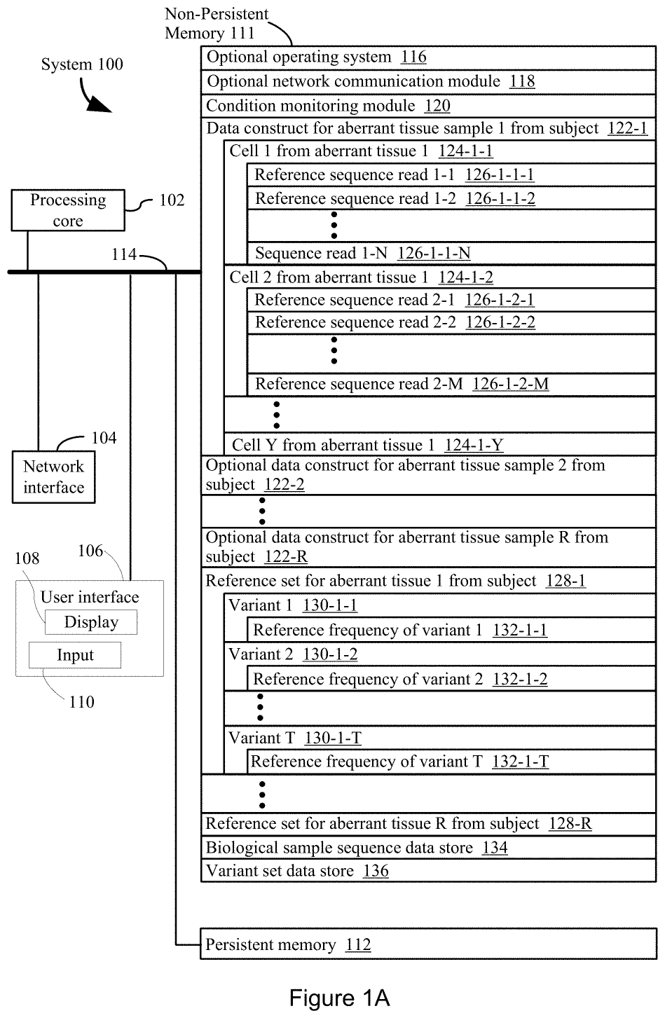

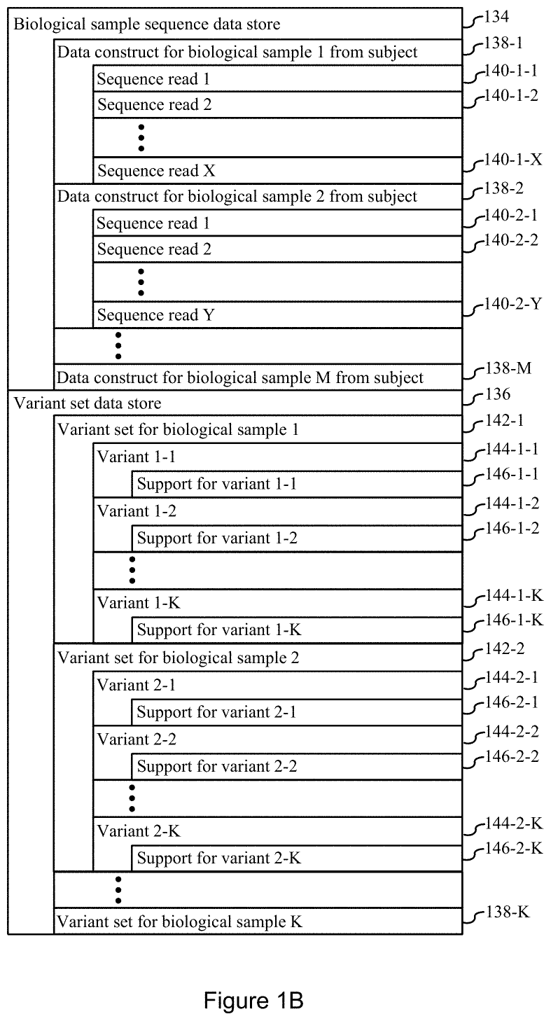

Now that an overview of some aspects of the present disclosure and some definitions used in the present disclosure have been provided, details of an exemplary system are now described in conjunction with FIG. 1. FIG. 1 is a block diagram illustrating a system 100 in accordance with some implementations. The device 100 in some implementations includes one or more processing units CPU(s) 102 (also referred to as processors), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an operating system 116, which includes procedures for handling various basic system services and for performing hardware dependent tasks;
a network communication module (or instructions) 118 for connecting the system 100 with other devices, or a communication network;
a variable selection module 120 for identifying features informative of a phenotype of interest;
a raw data normalization module 122 for normalizing raw feature data 136 within each raw training dataset 132;
a data co-normalization module 124 for co-normalizing feature data, e.g., normalized feature data 142, across heterogeneous training datasets, e.g., internally normalized data constructs 138;
a classifier training module 126 for training a machine learning classifier based on co-normalized feature data 148 across heterogeneous datasets;
a training dataset store 130 for storing one or more data constructs, e.g., raw data constructs 132, internally normalized data constructs 138, and/or co-normalized data constructs 144 for one or more samples from training subjects, each such data construct including for each respective training subject in a plurality of training subjects, a plurality of feature values, e.g., raw feature values 136, internally normalized feature values 142, and/or co-normalized feature values 148;
a data module set store 150 for storing one or more modules 152 for training a classifier, each such respective module 150 including (i) an identification of an independent plurality of differentially-regulated features 154, (ii) a corresponding summarization algorithm or component classifier 156, and (iii) an independent phenotype 157 associated with a clinical condition under study (e.g., the clinical condition itself or a phenotype that is dispositive or associated with the clinical condition); and
a test dataset store 160 for storing one or more data constructs 162 for one or more samples from test subjects 164, each such data construct including a plurality of feature values 166.

In some implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

### Exemplary Method Embodiment

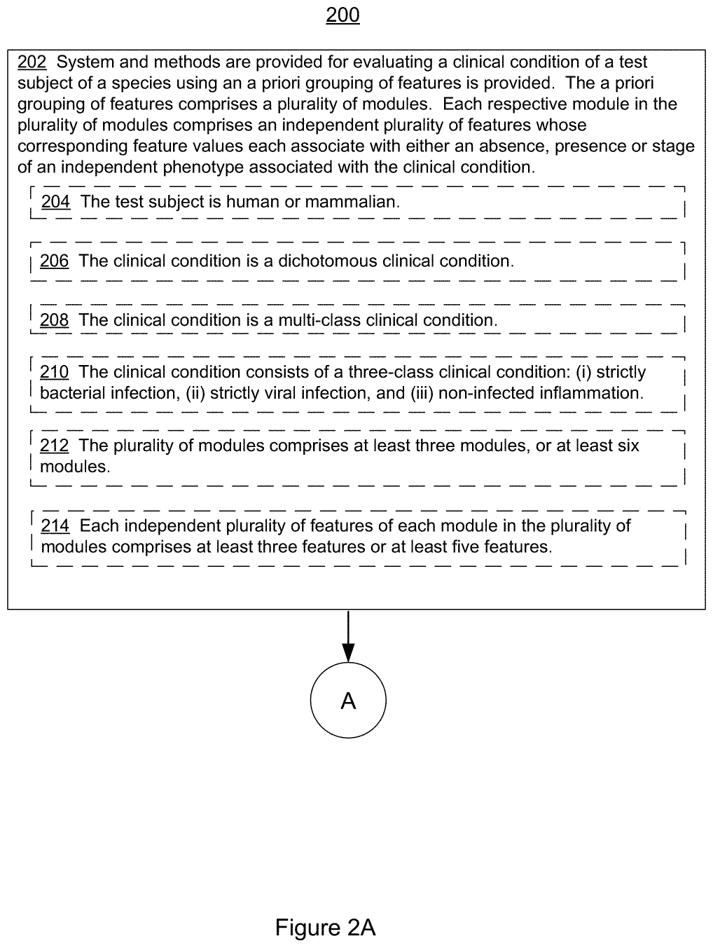

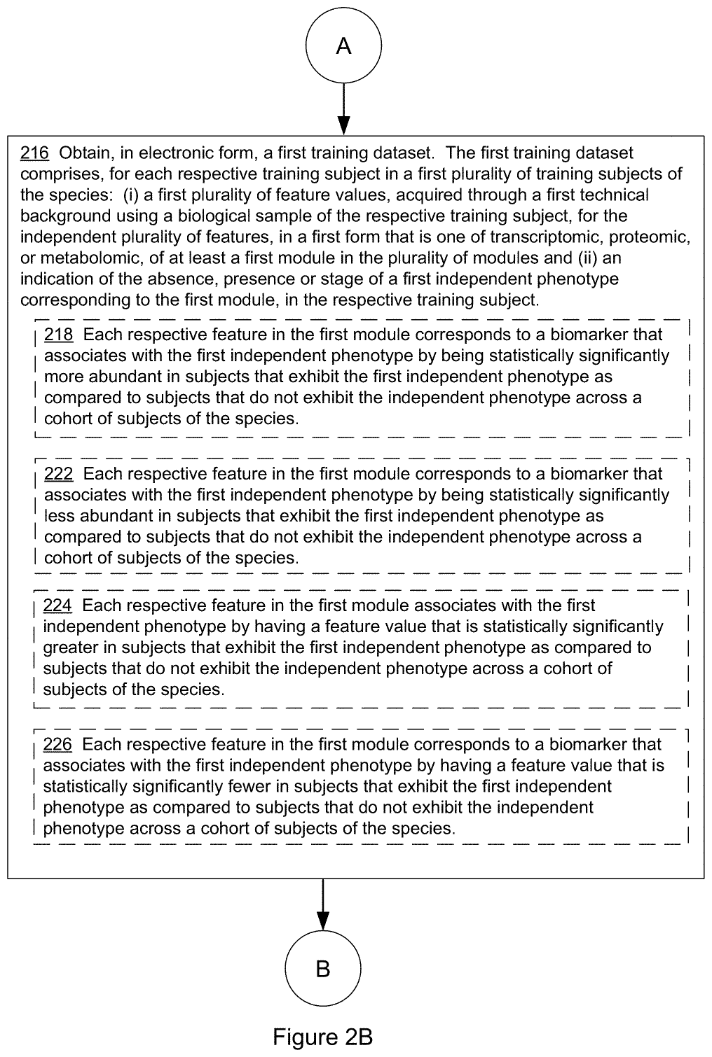

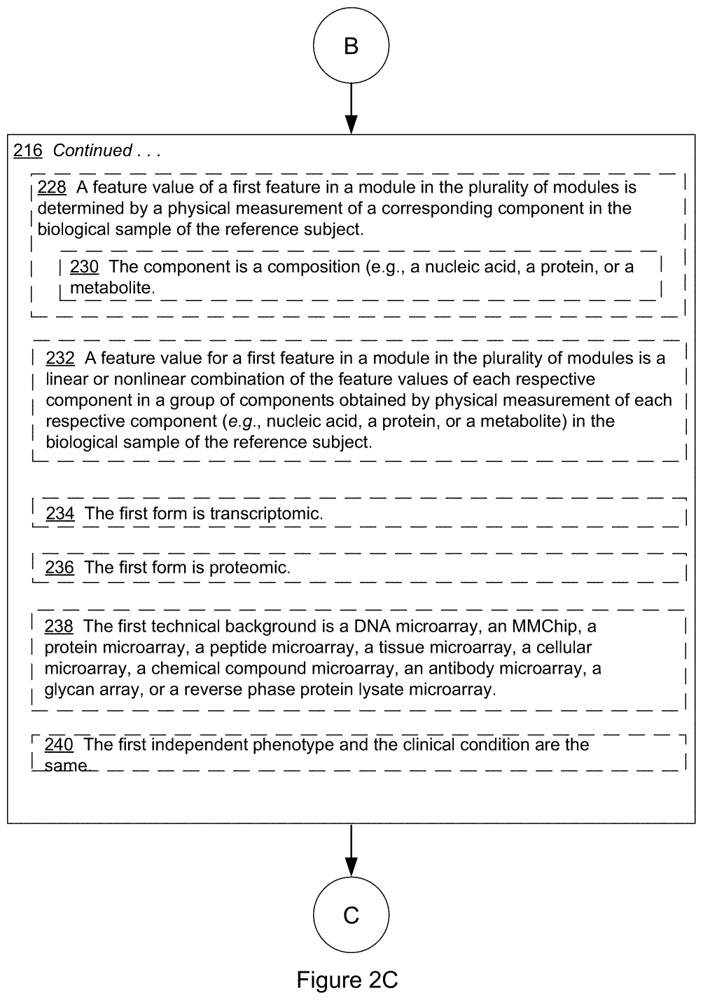

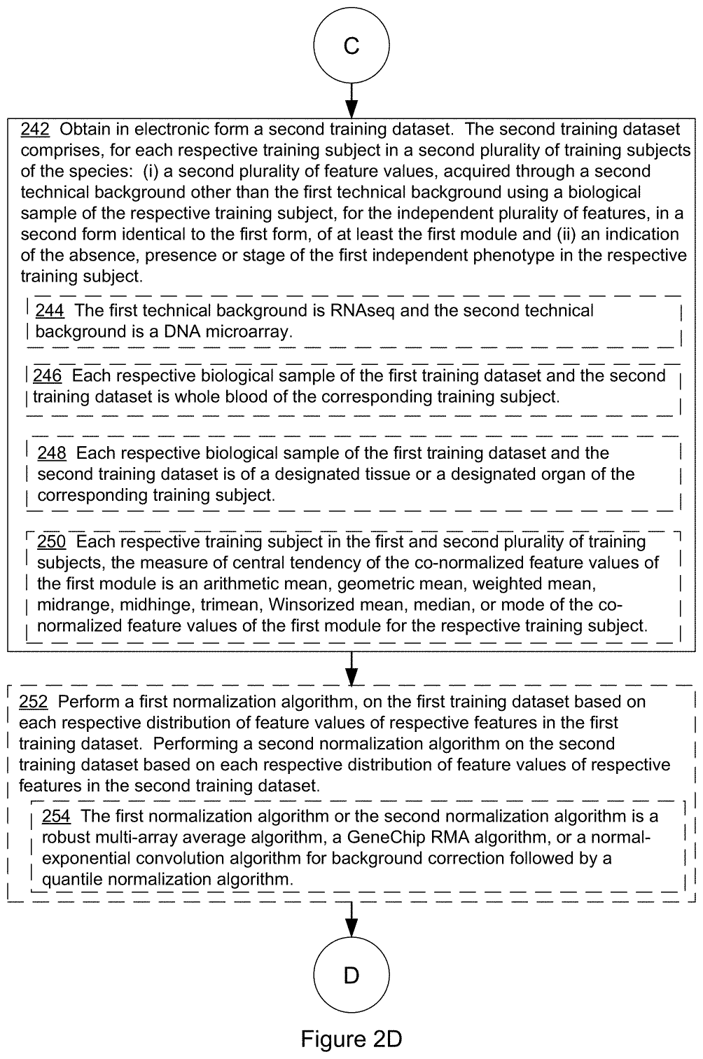

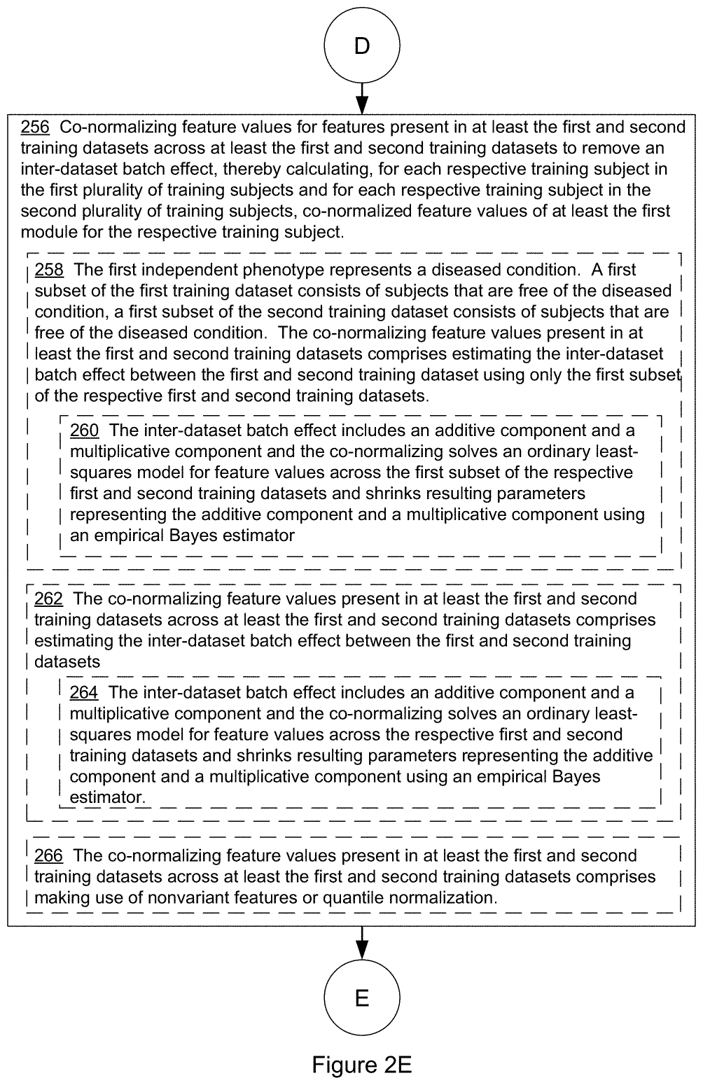

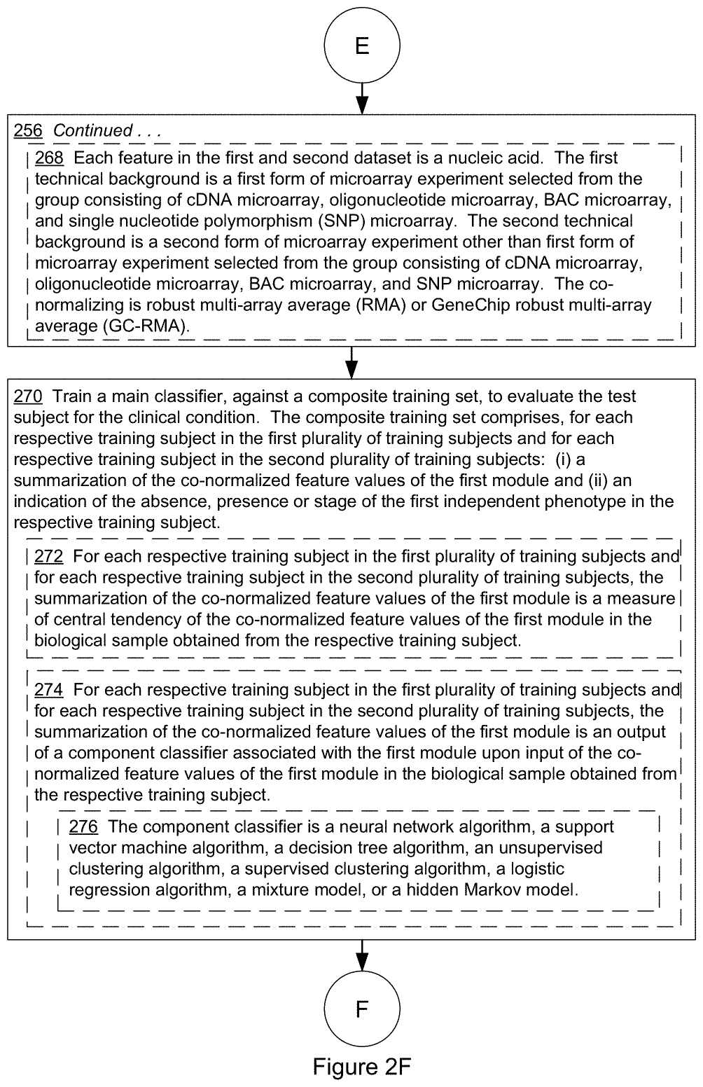

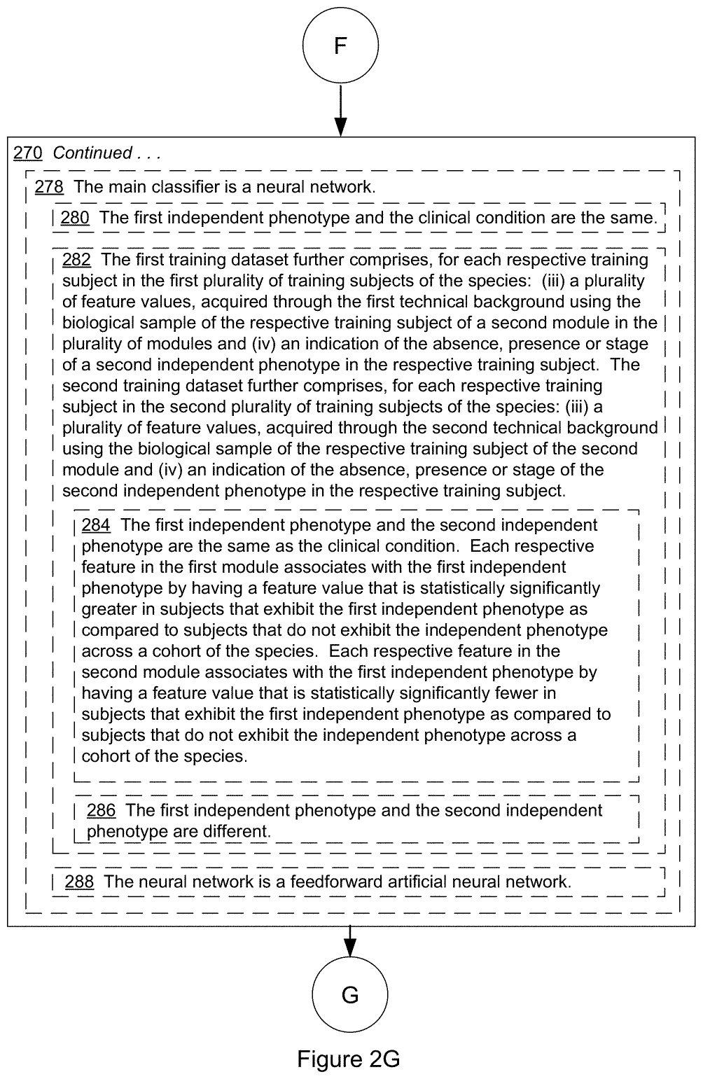

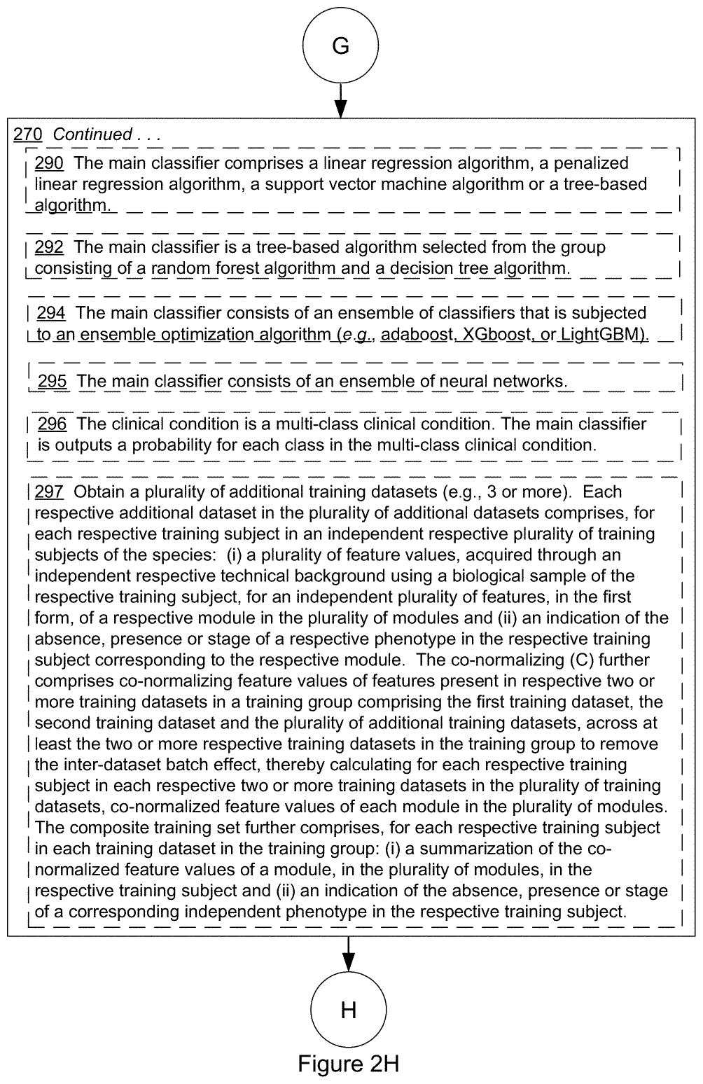

While a system in accordance with the present disclosure has been disclosed with reference to FIG. 1, a method in accordance with the present disclosure is now detailed with reference to FIG. 2.

Referring to blocks 202-214 of FIG. 2A, in some embodiments a method of evaluating a clinical condition of a test subject of a species using an a priori grouping of features is provided at a computer system, such as system 100 of FIG. 1, which has one or more processors 102 and memory 111/112 storing one or more programs, such as variable selection module 120, for execution by the one or more processors. The a priori grouping of features comprises a plurality of modules 152. Each respective module 152 in the plurality of modules 152 comprises an independent plurality of features 154 whose corresponding feature values each associate with either an absence, presence or stage of an independent phenotype 157 associated with the clinical condition. For example, Table 1 provides a non-limiting example definition and composition of six sepsis-related modules (sets of genes) that are each associated with an absence, presence or stage of an independent phenotype 157 associated with sepsis. Modules 152-1 and 152-2 of Table 1 are respectively are directed to the genes with elevated (module 152-1) and reduced (module 152-2) expression in strictly viral infection. Modules 152-3 and 152-4 of Table 1 are respectively directed to the genes with elevated (module 152-3) and reduced (module 152-4) expression in patients with sepsis versus sterile inflammation. Modules 152-5 and 152-6 are respectively directed to genes with elevated (module 152-5) and reduced (module 152-6) expression in patients who died within 30 days of hospital admission.

TABLE 1
Definition and composition of sepsis-related modules

| Module | | Differentially-regulated features |
|---|---|---|
| Number | Phenotype | 154 |
| 152-1 | Fever-up | IFI27, JUP, LAX1 |
| 152-2 | Fever-down | HK3, TNIP1, GPAA1, CTSB |
| 152-3 | Sepsis-up | CEACAM1, ZDHHC19, C9orf95, |
| | | GNA15, BATF, C3AR1 |
| 152-4 | Sepsis-down | KIAA1370, TGFBI, MTCH1, |
| | | RPGRIP1, HLA-DPB1 |
| 152-5 | Severity-up | DEFA4, CD163, RGS1, PER1, |
| | | HIF1A, |
| | | SEPP1, C11orf74, CIT |
| 152-6 | Severity-down | LY86, TST, KCNJ2 |

Referring to block 204, in some embodiments the subject is human or mammalian. In some embodiment, the subject is any living or non-living organism, including but not limited to a human (e.g., a male human, female human, fetus, pregnant female, child, or the like), a non-human animal, a plant, a bacterium, a fungus or a protist. In some embodiments, subject is a mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

Referring to block 206, in some embodiments, the clinical condition is a dichotomous clinical condition (e.g, has sepsis versus does not have sepsis, has cancer versus does not have cancer, etc.). Referring to block 208, in some embodiments, the clinical condition is a multi-class clinical condition. For example, referring to block 210, in some embodiments, the clinical condition consists of a three-class clinical condition: (i) strictly bacterial infection, (ii) strictly viral infection, and (iii) non-infected inflammation.

Referring to block 212, in some embodiments, the plurality of modules 152 comprises at least three modules, or at least six modules. Table 1 above provides an example in which the plurality of modules 152 consists of six modules. In some embodiments, the plurality of modules 152 comprises between three and one hundred modules. In some embodiments, the plurality of modules 152 consists of two modules.

Moreover, referring to block 214, in some embodiments, each independent plurality of features 154 of each module 152 in the plurality of modules comprises at least three features or at least five features. Table 1 above provides an example in which the plurality of modules 152 consists of six modules. In some embodiments, the plurality of modules 152 comprises between three and one hundred modules. In some embodiments, the plurality of modules 152 consists of two modules. Moreover, there is no requirement that each module include the same number of features. This is demonstrated by the example of Table 1 above. Thus, for example, in some embodiments, one module 152 can have two features 154 while another module can have over fifty features. In some embodiments, each module 152 has between two and fifty features 154. In some embodiments, each module 152 has between three and one hundred features. In some embodiments, each module 152 has between four and two hundred features. In some embodiments, the features 154 in each module 152 are unique. That is, any given feature only appears in one of the modules 152. In still other embodiments, there is no requirement that the features in each module 152 be unique, that is, a given feature 154 can be in more than one module in such embodiments.

Referring to block 216 of FIG. 2B, a first training dataset (e.g., raw data construct 132-1 of FIG. 1A) is obtained. The first training dataset comprises, for each respective training subject 134 in a first plurality of training subjects of the species: (i) a first plurality of feature values 136, acquired through a first technical background using a biological sample of the respective training subject, for the independent plurality of features, in a first form that is one of transcriptomic, proteomic, or metabolomic, of at least a first module 152 in the plurality of modules and (ii) an indication of the absence, presence or stage of a first independent phenotype 157 corresponding to the first module, in the respective training subject. In practice, because this is a training dataset, the dataset will provide an indication of the clinical condition of each subject. However, in some embodiments, the first independent phenotype and the clinical condition are one in the same. In embodiments where they are not one in the same, the training set provides both the first independent phenotype and the clinical condition. For example, in the case where the first module is module 152-1 of Table 1 above, the first dataset will provide for each respective training subject in the first dataset: (i) measured expression values for the genes IFI27, JUP, and LAX1, acquired through a first technical background using a biological sample of the respective training subject, (ii) an indication as to whether the subject has fever, and (iii) whether the subject has sepsis.

In some embodiments, each module 158 is uniquely associated with an absence, presence or stage of an independent phenotype associated with the clinical condition but the first training dataset only provides an indication of the absence, presence or stage of the clinical condition itself, not the independent phenotype 157 of each respective module, for each training subject. For example, in the case of Table 1, in some embodiments, the first training dataset includes an indication of the absence, presence or stage of the clinical condition (sepsis), but does not indicate whether each training subject has the phenotype fever. That is, in some embodiments, the present disclosure relies on previous work that has identified which features are upregulated or downregulated with respect to the given phenotype, such as fever, and thus an indication of whether each training subject in the training dataset has the phenotype of the module is not necessary. In instances, where the phenotype corresponding to a module is not provided, an indication as to the absence, presence or stage of the clinical condition in the training subjects is provided.

In some embodiments, the first training dataset only provides the absence or presence of a clinical condition for each training subject. That is, stage of the clinical condition is not provided in such embodiments.

Referring to block 218 of FIG. 2B, in some embodiments, each respective feature in the first module corresponds to a biomarker that associates with the first independent phenotype by being statistically significantly more abundant in subjects that exhibit the first independent phenotype as compared to subjects that do not exhibit the independent phenotype across a cohort of subjects of the species. The cohort of subjects of the species need not be the subjects of the first dataset. The cohort of subjects of the species is any groups of subjects that meet selection criteria and that include subjects that have the clinical condition and subjects that do not have the clinical condition. Nonlimiting example selection criteria for the cohort in the case of sepsis are: 1) are physician-adjudicated for the presence and type of infection (e.g. strictly bacterial infection, strictly viral infection, or non-infected inflammation), 2) have feature values for the features in the plurality of modules, 3) were over 18 years of age, 4) were seen in hospital settings (e.g. emergency department, intensive care), 5) were either community- or hospital-acquired infection, and 6) had blood samples taken within 24 hours of initial suspicion of infection and/or sepsis. In some such embodiments, the determination as to whether a biomarker is “statistically significantly more abundant” is evaluated by applying a standard t-test, Welch t-test, Wilcoxon test, or permutation test to the abundance of the biomarker as measured in subjects in the cohort that exhibit the first independent phenotype (group 1) and subjects in the cohort that do not exhibit the first independent phenotype (group 2) to arrive at a p-value. In some such embodiments, a biomarker is statistically significantly more abundant when the p-value in such a test is 0.05 or less, 0.005 or less, or 0.001 or less. In some such embodiments, a biomarker is statistically significantly more abundant when the p-value in such a test is 0.05 or less, 0.005 or less, or 0.001 or less adjusted for multiple testing using a False Discovery Rate procedure such as Benjamini-Hochberg or Benjamini-Yekutieli. See, for example, Benjamini and Hochberg, Journal of the Royal Statistical Society, Series B 57, pp. 289-300; and Benjamini and Yekutieli, 2005, Journal of American Statistical Association 100(469), pp. 71-80, each of which is hereby incorporated by reference. In some embodiments, a biomarker is deemed to be statistically significantly more abundant via fixed-effects or random-effects meta-analysis of multiple datasets (cohorts or training datasets). See, for example, Sianphoe et al., 2019, BMC Bioinformatics 20:18, which is hereby incorporated by reference.

In some embodiments, each module 152 is uniquely associated with an absence, presence or stage of an independent phenotype 157 associated with the clinical condition but the first training dataset only provides an indication of the absence, presence or stage of the clinical condition itself, and the absence, presence or stage of the independent phenotype of some but not all of the plurality of modules, for each training subject in the first training set. For example, in the case of Table 1, in some embodiments, the first training dataset includes an indication of the absence, presence or stage of the clinical condition/phenotype “sepsis,” an indication of the absence, presence or stage of the phenotype “severity,” but does not indicate whether each training subject has fever.

Referring to block 222 of FIG. 2B, in some embodiments, each respective feature in the first module corresponds to a biomarker that associates with the first independent phenotype 157 by being statistically significantly less abundant in subjects that exhibit the first independent phenotype as compared to subjects that do not exhibit the independent phenotype across a cohort of subjects of the species. In some embodiments, the determination as to whether a biomarker is “statistically significantly less abundant” is evaluated by applying a standard t-test, Welch t-test, Wilcoxon test, or permutation test to the abundance of the biomarker as measured in subjects in the cohort that exhibit the first independent phenotype (group 1) and subjects in the cohort that do not exhibit the first independent phenotype (group 2) to arrive at a p-value. In some such embodiments, a biomarker is statistically significantly less abundant when the p-value in such a test is 0.05 or less, 0.005 or less, or 0.001 or less. In some such embodiments, a biomarker is statistically significantly less abundant when the p-value in such a test is 0.05 or less, 0.005 or less, or 0.001 or less adjusted for multiple testing using a False Discovery Rate procedure such as Benjamini-Hochberg or Benjamini-Yekutieli. See, for example, Benjamini and Hochberg, Journal of the Royal Statistical Society, Series B 57, pp. 289-300; and Benjamini and Yekutieli, 2005, Journal of American Statistical Association 100(469), pp. 71-80, each of which is hereby incorporated by reference. In some embodiments, a biomarker is deemed to be statistically significantly less abundant via fixed-effects or random-effects meta-analysis of multiple datasets (cohorts or training datasets). See, for example, Sianphoe et al., 2019, BMC Bioinformatics 20:18, which is hereby incorporated by reference.

Referring to block 224 of FIG. 2B, in some embodiments, each respective feature in the first module associates with the first independent phenotype 157 by having a feature value that is statistically significantly greater in subjects that exhibit the first independent phenotype as compared to subjects that do not exhibit the independent phenotype across a cohort of subjects of the species. In some embodiments, the determination as to whether a feature is “statistically significantly more abundant” is evaluated by applying a standard t-test, Welch t-test, Wilcoxon test, or permutation test to the abundance of the feature as measured in subjects in the cohort that exhibit the first independent phenotype (group 1) and subjects in the cohort that do not exhibit the first independent phenotype (group 2) to arrive at a p-value. In some such embodiments, a feature value is statistically significantly greater when the p-value in such a test is 0.05 or less, 0.005 or less, or 0.001 or less. In some such embodiments, a feature is statistically significantly greater (more abundant) when the p-value in such a test is 0.05 or less, 0.005 or less, or 0.001 or less adjusted for multiple testing using a False Discovery Rate procedure such as Benjamini-Hochberg or Benjamini-Yekutieli. See, for example, Benjamini and Hochberg, Journal of the Royal Statistical Society, Series B 57, pp. 289-300; and Benjamini and Yekutieli, 2005, Journal of American Statistical Association 100(469), pp. 71-80, each of which is hereby incorporated by reference. In some embodiments, a feature is deemed to be statistically significantly greater via fixed-effects or random-effects meta-analysis of multiple datasets (cohorts or training datasets). See, for example, Sianphoe et al., 2019, BMC Bioinformatics 20:18, which is hereby incorporated by reference.

Referring to block 226 of FIG. 2B, in some embodiments, each respective feature in the first module associates with the first independent phenotype 157 by having a feature value that is statistically significantly fewer in subjects that exhibit the first independent phenotype as compared to subjects that do not exhibit the independent phenotype across a cohort of subjects of the species. In some embodiments, the determination as to whether a feature is “statistically significantly fewer” is evaluated by applying a standard t-test, Welch t-test, Wilcoxon test, or permutation test to the abundance of the feature as measured in subjects in the cohort that exhibit the first independent phenotype (group 1) and subjects in the cohort that do not exhibit the first independent phenotype (group 2) to arrive at a p-value. In some such embodiments, a feature is statistically significantly fewer when the p-value in such a test is 0.05 or less, 0.005 or less, or 0.001 or less. In some such embodiments, a feature is statistically significantly fewer when the p-value in such a test is 0.05 or less, 0.005 or less, or 0.001 or less adjusted for multiple testing using a False Discovery Rate procedure such as Benjamini-Hochberg or Benjamini-Yekutieli. See, for example, Benjamini and Hochberg, Journal of the Royal Statistical Society, Series B 57, pp. 289-300; and Benjamini and Yekutieli, 2005, Journal of American Statistical Association 100(469), pp. 71-80, each of which is hereby incorporated by reference. In some embodiments, a feature is deemed to be statistically significantly fewer via fixed-effects or random-effects meta-analysis of multiple datasets (cohorts or training datasets). See, for example, Sianphoe et al., 2019, BMC Bioinformatics 20:18, which is hereby incorporated by reference.

Referring to block 228 of FIG. 2C, in some embodiments, a feature value of a first feature in a module 152 in the plurality of modules is determined by a physical measurement of a corresponding component in the biological sample of the reference subject. Referring to block 230, examples of components, include but are not limited to, compositions (e.g., a nucleic acid, a protein, or a metabolite).

Referring to block 232 of FIG. 2C, in some embodiments, a feature value for a first feature in a module 152 in the plurality of modules is a linear or nonlinear combination of the feature values of each respective component in a group of components obtained by physical measurement of each respective component (e.g., nucleic acid, a protein, or a metabolite) in the biological sample of the reference subject.

It was noted with respect to block 216 that the first training set was obtained using a biological sample of the respective training subject, for the independent plurality of features, in a first form that is one of transcriptomic, proteomic, or metabolomics. Referring to block 234, in some embodiments the first form is transcriptomic. Referring to block 236, in some embodiments the first form is proteomic.

It was noted with respect to block 216 that the first training set comprises a first plurality of feature values, acquired through a first technical background, for each respective training subject in a first plurality of training subjects. Referring to block 238, in some embodiments this first technical background is a DNA microarray, an MMChip, a protein microarray, a peptide microarray, a tissue microarray, a cellular microarray, a chemical compound microarray, an antibody microarray, a glycan array, or a reverse phase protein lysate microarray.

In some embodiments, the biological sample collected from each subject is blood, whole blood, plasma, serum, urine, cerebrospinal fluid, fecal, saliva, sweat, tears, pleural fluid, pericardial fluid, or peritoneal fluid of the subject. In some embodiments, the biological sample is a specific tissue of the subject. In some embodiments, the biological sample is a biopsy of a specific tissue or organ (e.g., breast, lung, prostate, rectum, uterus, pancreas, esophagus, ovary, bladder, etc.) of the subject.

In some embodiments, the features are nucleic acid abundance values for nucleic acids corresponding to genes of the species that is obtained from sequencing sequence reads that are, in turn, from nucleic acids in the biological sample and represent the abundance of such nucleic acids, and the genes they represent, in the biological same. Any form of sequencing can be used to obtain the sequence reads from the nucleic acid obtained from the biological sample including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads 140 from the cell-free nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain sequence reads from the nucleic acid obtained from the biological sample. In some such embodiments, millions of cell-free nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instance, flow cells are planar in shape, optically transparent, generally in the millimeter or sub-millimeter scale, and often have channels or lanes in which the analyte/reagent interaction occurs. In some embodiments, a cell-free nucleic acid sample can include a signal or tag that facilitates detection. In some such embodiments, the acquisition of sequence reads from the nucleic acid obtained from the biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

Referring to block 240, in some embodiments the first independent phenotype of a module and the clinical condition are the same. This is illustrated for modules 152-3 and 152-4 of Table 1 in which the clinical condition is sepsis and the first independent phenotype of module 152-3 is “sepsis-down” and the first independent phenotype of module 152-4 is sepsis-down. Thus, for modules 152-3 and 152-4, all that is necessary in the training set (other than the feature value abundances) is for each training subject to be labeled as having sepsis or not.

Referring to block 242, in some embodiments a second training dataset is obtained. The second training dataset comprises, for each respective training subject in a second plurality of training subjects of the species: (i) a second plurality of feature values, acquired through a second technical background other than the first technical background using a biological sample of the respective training subject, for the independent plurality of features, in a second form identical to the first form, of at least the first module and (ii) an indication of the absence, presence or stage of the first independent phenotype in the respective training subject.

Referring to block 244, in some embodiments, the first technical background (through which the first training set is acquired) is RNAseq and the second technical background (through which the second training set is acquired) is a DNA microarray.

In some embodiments, the first technical background is a first form of microarray experiment selected from the group consisting of cDNA microarray, oligonucleotide microarray, BAC microarray, and single nucleotide polymorphism (SNP) microarray and the second technical background is a second form of microarray experiment other than first form of microarray experiment selected from the group consisting of cDNA microarray, oligonucleotide microarray, BAC microarray, and SNP microarray.

In some embodiments, the first technical background is nucleic acid sequencing using the sequencing technology of a first manufacturer and the second technical background is nucleic acid sequencing using the sequencing technology of a second manufacturer (e.g., an Illumina beadchip versus an Affymetrix or Agilent microarray).

In some embodiments, the first technical background is nucleic acid sequencing using a first sequencing instrument to a first sequencing depth and the second technical background is nucleic acid sequencing using a second sequencing instrument to a second sequencing depth, where the first sequencing depth is other than the second sequencing depth and the first sequencing instrument is the same make and model as the second sequencing instrument but the first and second instruments are different instruments.

In some embodiments, the first technical background is a first type of nucleic acid sequencing (e.g., microarray based sequencing) and the second technical background is a second type of nucleic acid sequencing other than the first type of nucleic acid sequencing (e.g., next generation sequencing).

In some embodiments, the first technical background is paired end nucleic acid sequencing and the second technical background is single read nucleic acid sequencing.

The above are nonlimiting examples of different technical backgrounds. In general, two technical backgrounds are different when the feature abundance data is captured under different technical conditions, such as different machines, different methods, or under different technical conditions, such as different reagents, or under different technical parameters (e.g., in the case of nucleic acid sequencing, different coverages, etc.).

Referring to block 248, in some embodiments, each respective biological sample of the first training dataset and the second training dataset is of a designated tissue or a designated organ of the corresponding training subject. For example, in some embodiments each biological sample is a blood sample. In another example, each biological sample is a breast biopsy, lung biopsy, prostate biopsy, rectum biopsy, uterine biopsy, pancreatic biopsy, esophagus biopsy, ovary biopsy, or bladder biopsy.

Referring to block 252 of FIG. 2D, in some embodiments, a first normalization algorithm is performed on the first training dataset based on each respective distribution of feature values of respective features in the first training dataset. Further, a second normalization algorithm on the second training dataset based on each respective distribution of feature values of respective features in the second training dataset. Referring to block 254 of FIG. 2D, in some embodiments, the first normalization algorithm or the second normalization algorithm is a robust multi-array average algorithm, a GeneChip RMA algorithm, or a normal-exponential convolution algorithm for background correction followed by a quantile normalization algorithm.

In some embodiments, such normalization is not performed in the disclosed methods. As a non-limiting example, in such embodiments the normalization of block 252 is not performed because the datasets are already normalized. As another non-limiting example, in some embodiments the normalization of block 252 is not performed because such normalization is determined to not be necessary.

Referring to block 256, feature values for features present in at least the first and second training datasets are co-normalized across at least the first and second training datasets to remove an inter-dataset batch effect, thereby calculating, for each respective training subject in the first plurality of training subjects and for each respective training subject in the second plurality of training subjects, co-normalized feature values of at least the first module for the respective training subject. In some such embodiments, such normalization provides co-normalized feature values of each of the plurality of modules for the respective training subject.

Referring to block 258, in some embodiments, the first independent phenotype (of the first module) represents a diseased condition. Further, a first subset of the first training dataset consists of subjects that are free of the diseased condition and a first subset of the second training dataset consists of subjects that are free of the diseased condition. Moreover, the co-normalizing of feature values present in at least the first and second training datasets comprises estimating the inter-dataset batch effect between the first and second training dataset using only the first subset of the respective first and second training datasets. Referring to block 260, in some such embodiments, the inter-dataset batch effect includes an additive component and a multiplicative component and the co-normalizing solves an ordinary least-squares model for feature values across the first subset of the respective first and second training datasets and shrinks resulting parameters representing the additive component and a multiplicative component using an empirical Bayes estimator. See, for example, Sweeney et al., 2016, Sci Transl Med 8(346), pp. 346ra91, which is hereby incorporated by reference.

Referring to block 264, in some embodiments, the co-normalizing of feature values present in at least the first and second training datasets across at least the first and second training datasets comprises estimating the inter-dataset batch effect between the first and second training datasets. Referring to block 266, in some embodiments, the inter-dataset batch effect includes an additive and a multiplicative component and the co-normalizing solves an ordinary least-squares model for feature values across the respective first and second training datasets and shrinks resulting parameters representing the additive component and a multiplicative component using an empirical Bayes estimator. See, for example, Sweeney et al., 2016, Sci Transl Med 8(346), pp. 346ra91, which is hereby incorporated by reference.

Referring to block 266 of FIG. 2E, in some embodiments, the co-normalizing feature values present in at least the first and second training datasets across at least the first and second training datasets comprises making use of nonvariant features, quantile normalization, or rank normalization. See Qiu et al., 2013, BMC Bioinformatics 14, p. 124; and Hendrik et al., 2007, PLoS One 2(9), p. e898, each of which is hereby incorporated by reference.

Referring to block 258 of FIG. 2F, in some embodiments, each feature in the first and second dataset is a nucleic acid. The first technical background is a first form of microarray experiment selected from the group consisting of cDNA microarray, oligonucleotide microarray, BAC microarray, and single nucleotide polymorphism (SNP) microarray. The second technical background is a second form of microarray experiment other than first form of microarray experiment selected from the group consisting of cDNA microarray, oligonucleotide microarray, BAC microarray, and SNP microarray. See, for example, Bumgarner, 2013, Current protocols in molecular biology, Chapter 22, which is hereby incorporated by reference. In some such embodiments, the co-normalizing is robust multi-array average (RMA), GeneChip robust multi-array average (GC-RMA), MASS, Probe Logarithmic Intensity ERror (Plier), dChip, or chip calibration. See, for example, Irizarry, 2003, Biostatistics 4(2), pp. 249-264; Welsh et al. 2013, BMC Bioinformatics 14, p. 153; and Therneau and Ballman, 2008, Cancer Inform 6, pp. 423-431; and Oberg, 2006, Bioinformatics 22, pp. 2381-2387, each of which is hereby incorporated by reference.

Referring to FIG. 2F, the method continues with the training of a main classifier, against a composite training set, to evaluate the test subject for the clinical condition. The composite training set comprises, for each respective training subject in the first plurality of training subjects and for each respective training subject in the second plurality of training subjects: (i) a summarization of the co-normalized feature values of the first module and (ii) an indication of the absence, presence or stage of the first independent phenotype in the respective training subject.

Referring to block 270, in some such embodiments, for each respective training subject in the first and second plurality of training subjects, the summarization of the co-normalized feature values of the first module is a measure of central tendency (e.g., arithmetic mean, geometric mean, weighted mean, midrange, midhinge, trimean, Winsorized mean, median, or mode) of the co-normalized feature values of the first module in the biological sample obtained from the respective training subject. For instance, in some such embodiments, for each respective training subject in the first and second plurality of training subjects, the summarization of the co-normalized feature values of the first module is a measure of central tendency (e.g., arithmetic mean, geometric mean, weighted mean, midrange, midhinge, trimean, Winsorized mean, median, or mode) of the co-normalized feature values of each respective modules in the plurality of module, in the biological sample obtained from the respective training subject. This is illustrated in FIG. 3 in which each of modules fᵤₚ, f_{dn}, mᵤₚ, m_{dn}, sᵤₚ, and s_{dn} separately provides a measure of central tendency of their respective co-normalized feature values for a given training subject.

Referring to block 274, in alternative embodiments, for each respective training subject in the first plurality of training subjects and for each respective training subject in the second plurality of training subjects, the summarization of the co-normalized feature values of the first module is an output of a component classifier associated with the first module upon input of the co-normalized feature values of the first module in the biological sample obtained from the respective training subject. This is illustrated in FIG. 4, in which a mini ‘spoke’ of networks is used for each module. Individual features are summarized by a local network (instead of summarized by their geometric mean) and then passed into the main classification network (the main classifier). Referring to block 276, in some embodiments, the component classifier is a neural network algorithm, a support vector machine algorithm, a decision tree algorithm, an unsupervised clustering algorithm, a supervised clustering algorithm, a logistic regression algorithm, a mixture model, or a hidden Markov model.

As used herein, a main classifier refers to a model with fixed (locked) parameters (weights) and thresholds, ready to be applied to previously unseen samples (e.g., the test subject). In this context, a model refers to a machine learning algorithm, such as logistic regression, neural network, decision tree etc. (similar to models in statistics). Thus, referring to block 278 of FIG. 2G, in some embodiments, the main classifier is a neural network. That is, in such embodiments, the main classifier is a neural network with fixed (locked) parameters (weights) and thresholds. In some such embodiments, referring to block 280, the first independent phenotype and the clinical condition are the same.

Referring to block 282, in some embodiments in which the main classifier is a neural network, the first training dataset further comprises, for each respective training subject in the first plurality of training subjects of the species: (iii) a plurality of feature values, acquired through the first technical background using the biological sample of the respective training subject of a second module in the plurality of modules and (iv) an indication of the absence, presence or stage of a second independent phenotype in the respective training subject. The second training dataset further comprises, for each respective training subject in the second plurality of training subjects of the species: (iii) a plurality of feature values, acquired through the second technical background using the biological sample of the respective training subject of the second module and (iv) an indication of the absence, presence or stage of the second independent phenotype in the respective training subject. In other words, as illustrated in FIGS. 3 and 4, there can be more than one module. In the case of block 282, there are two modules. In accordance with block 284, in some such embodiments, the first independent phenotype and the second independent phenotype are the same as the clinical condition (e.g., sepsis). Each respective feature in the first module associates with the first independent phenotype by having a feature value that is statistically significantly greater in subjects that exhibit the first independent phenotype as compared to subjects that do not exhibit the independent phenotype across a cohort of the species. This is illustrated in FIG. 3 as the module mᵤₚ. In some embodiments, the determination as to whether a feature is “statistically significantly greater” is evaluated by applying a standard t-test, Welch t-test, Wilcoxon test, or permutation test to the abundance of the feature as measured in subjects in the cohort that exhibit the first independent phenotype (group 1) and subjects in the cohort that do not exhibit the first independent phenotype (group 2) to arrive at a p-value. In some such embodiments, a feature is statistically significantly fewer (less abundant) when the p-value in such a test is 0.05 or less, 0.005 or less, or 0.001 or less. In some such embodiments, a feature is statistically significantly fewer when the p-value in such a test is 0.05 or less, 0.005 or less, or 0.001 or less adjusted for multiple testing using a False Discovery Rate procedure such as Benjamini-Hochberg or Benjamini-Yekutieli. See, for example, Benjamini and Hochberg, Journal of the Royal Statistical Society, Series B 57, pp. 289-300; and Benjamini and Yekutieli, 2005, Journal of American Statistical Association 100(469), pp. 71-80, each of which is hereby incorporated by reference. In some embodiments, a feature is determined to be statistically significantly fewer via fixed-effects or random-effects meta-analysis of multiple datasets (cohorts or training datasets). See, for example, Sianphoe et al., 2019, BMC Bioinformatics 20:18, which is hereby incorporated by reference.

Each respective feature in the second module associates with the first independent phenotype by having a feature value that is statistically significantly fewer in subjects that exhibit the first independent phenotype as compared to subjects that do not exhibit the first independent phenotype across a cohort of the species. This is illustrated in FIG. 3 as the module m_{dn}. In some embodiments, the determination as to whether a feature is “statistically significantly fewer” is evaluated by applying a standard t-test, Welch t-test, Wilcoxon test, or permutation test to the abundance of the feature as measured in subjects in the cohort that exhibit the first independent phenotype (group 1) and subjects in the cohort that do not exhibit the first independent phenotype (group 2) to arrive at a p-value. In some such embodiments, a feature is statistically significantly fewer (less abundant) when the p-value in such a test is 0.05 or less, 0.005 or less, or 0.001 or less. In some such embodiments, a feature is statistically significantly fewer when the p-value in such a test is 0.05 or less, 0.005 or less, or 0.001 or less adjusted for multiple testing using a False Discovery Rate procedure such as Benjamini-Hochberg or Benjamini-Yekutieli. See, for example, Benjamini and Hochberg, Journal of the Royal Statistical Society, Series B 57, pp. 289-300; and Benjamini and Yekutieli, 2005, Journal of American Statistical Association 100(469), pp. 71-80, each of which is hereby incorporated by reference. In some embodiments, a feature is determined to be statistically significantly fewer via fixed-effects or random-effects meta-analysis of multiple datasets (cohorts or training datasets). See, for example, Sianphoe et al., 2019, BMC Bioinformatics 20:18, which is hereby incorporated by reference.

Referring to block 286, in some embodiments of the embodiment of block 282, the first independent phenotype and the second independent phenotype are different (e.g, as illustrated in FIG. 3 with module fᵤₚ versus module sᵤₚ).

Referring to block 288, in some embodiments, the neural network is a feedforward artificial neural network. See, for example, Svozil et al., 1997, Chemometrics and Intelligent Laboratory Systems 39(1), pp. 43-62, which is hereby incorporated by reference, for disclosure on feedforward artificial neural networks.

Referring to block 290 of FIG. 2H, in some embodiments, the main classifier comprises a linear regression algorithm or a penalized linear regression algorithm. See for example, Hastie et al., 2001, The Elements of Statistical Learning, Springer-Verlag, New York, for disclosure on linear regression algorithms and penalized linear regression algorithms.

In some embodiments, the main classifier is a neural network. See, for example, Hassoun, 1995, Fundamentals of Artificial Neural Networks, Massachusetts Institute of Technology, which is hereby incorporated by reference.

In some embodiments, the main classifier is a support vector machine algorithm. SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety.

In some embodiments, the main classifier is a tree-based algorithm (e.g., a decision tree). Referring to block 292 of FIG. 2H, in some embodiments, the main classifier is a tree-based algorithm selected from the group consisting of a random forest algorithm and a decision tree algorithm. Decision trees are described generally by Duda, 2001, Pattern Classification, John Wiley & Sons, Inc., New York, pp. 395-396, which is hereby incorporated by reference.

Referring to block 294 of FIG. 2H, in some embodiments, the main classifier consists of an ensemble of classifiers that is subjected to an ensemble optimization algorithm (e.g., adaboost, XGboost, or LightGBM). See Alafate and Freund, 2019, “Faster Boosting with Smaller Memory,” arXiv:1901.09047v1, which is hereby incorporated by reference

Referring to block 295 of FIG. 2H, in some embodiments, the main classifier consists of an ensemble of neural networks. See Zhou et al., 2002, Artificial Intelligence 137, pp. 239-263, which is hereby incorporated by reference.

Referring to block 296 of FIG. 2H, in some embodiments the clinical condition is a multi-class clinical condition and the main classifier outputs a probability for each class in the multi-class clinical condition. For instance, referring to FIG. 3, in some embodiments the clinical condition is a three-class condition of bacterial infection (I_{bac}), viral infection (Iᵥᵢᵣₐ) or a non-viral, non-bacterial based infection (Iₙₒₙ) and the classifier provides a probability that the subject has I_{bac}, a probability that the subject has Iᵥᵢᵣₐ, and a probability that the subject has Iₙₒₙ. (where the probabilities sum up to one hundred percent).

Referring to block 297, in some embodiments, a plurality of additional training datasets is obtained (e.g., 3 or more, 4 or more, 5 or more, 6 or more, 10 or more, or 30 or more). Each respective additional dataset in the plurality of additional datasets comprises, for each respective training subject in an independent respective plurality of training subjects of the species: (i) a plurality of feature values, acquired through an independent respective technical background using a biological sample of the respective training subject, for an independent plurality of features, in the first form, of a respective module in the plurality of modules and (ii) an indication of the absence, presence or stage of a respective phenotype in the respective training subject corresponding to the respective module. In such embodiments, the co-normalizing of block 256 further comprises co-normalizing feature values of features present in respective two or more training datasets in a training group comprising the first training dataset, the second training dataset and the plurality of additional training datasets, across at least the two or more respective training datasets in the training group to remove the inter-dataset batch effect, thereby calculating for each respective training subject in each respective two or more training datasets in the plurality of training datasets, co-normalized feature values of each module in the plurality of modules. Further, the composite training set further comprises, for each respective training subject in each training dataset in the training group: (i) a summarization of the co-normalized feature values of a module, in the plurality of modules, in the respective training subject and (ii) an indication of the absence, presence or stage of a corresponding independent phenotype in the respective training subject.

Referring to block 298, in some embodiments a test dataset comprising a plurality of feature values is obtained. The plurality of feature values is measured in a biological sample of the test subject, for features in at least the first module, in the first form (transcriptomic, proteomic, or metabolomics). The test dataset is inputted into the main classifier thereby evaluating the test subject for the clinical condition. That is, the main classifier, responsive to inputting the main classifier provides a determination of the clinical condition of the test subject. In some embodiments, the clinical condition is multi-class, as illustrated and FIG. 3 and the determination of the clinical condition of the test subject provided by the main classifier is a probability that the test subject has each component class in the multi-class clinical condition.

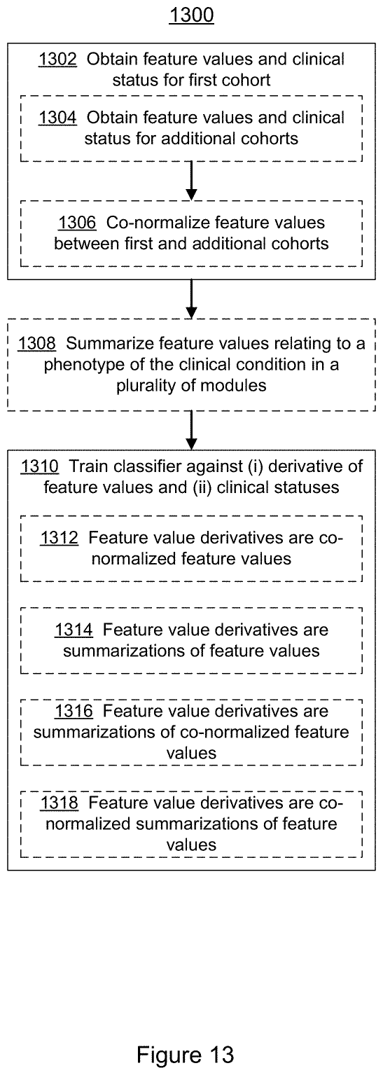

In some embodiments, the disclosure relates to a method 1300 for training a classifier for evaluating a clinical condition of a test subject, detailed below with reference to FIG. 13. In some embodiments, method 1300 is performed at a system as described herein, e.g., system 100 as described above with respect to FIG. 1. In some embodiments, method 1300 is performed at a system having a subset of the modules and/or data bases as described with respect to system 100.

Method 1300 includes obtaining (1302) feature values and clinical status for a first cohort of training subjects. In some embodiments, the feature values are collected from a biological sample from the training subjects in the first cohort, e.g., as described above with respect to method 200. Non-limiting examples of biological samples include solid tissue samples and liquid samples (e.g., whole blood or blood plasma samples). More details with respect to samples that are useful for method 1300 are described above with reference to method 200, and are not repeated here for brevity. In some embodiments, the methods described herein include a step of measuring the various feature values. In other embodiments, the methods described herein obtain, e.g., electronically, feature values that were previously measured, e.g., as stored in one or more clinical databases.

Two examples of measurement techniques include nucleic acid sequencing (e.g., qPCR or RNAseq) and microarray measurement (e.g., using a DNA microarray, an MMChip, a protein microarray, a peptide microarray, a tissue microarray, a cellular microarray, a chemical compound microarray, an antibody microarray, a glycan array, or a reverse phase protein lysate microarray). However, the skilled artisan will know of other measurement techniques for measuring features from a biological sample. More details with respect to feature measurement techniques (e.g., technical backgrounds) that are useful for method 1300 are described above with reference to method 200, and are not repeated here for brevity.

In some embodiments, the feature values for each training subject in the first cohort are collected using the same measurement technique. For example, in some embodiments, each of the features is of a same type, e.g., an abundance for a protein, nucleic acid, carbohydrate, or other metabolite, and the technique used to measure the feature values for each value is consistent across the first cohort. For instance, in some embodiments, the features are abundances of mRNA transcripts and the measuring technique is RNAseq or a nucleic acid microarray. In other embodiments, e.g., in some embodiments when feature values are not co-normalized across different cohorts of training subjects, different techniques are used to measure the feature values across the first cohort of training subject. However, in some embodiments where feature values are not co-normalized across different cohorts, e.g., where a single cohort of training subjects are used to train a classifier, the same technique is used to measure feature values across the first cohort.

In some embodiments, method 1300 includes obtaining (1304) feature values and clinical status for additional cohorts of training subjects. In some embodiments, feature values are collected for at least 2 additional cohorts. In some embodiments, feature values are collected for at least 3, 4, 5, 6, 7, 8, 9, 10, or more additional cohorts. In some embodiments, the feature values obtained for each cohort were measured using the same technique. That is, all the feature values obtained for the first cohort were measured using a first technique, all the feature values obtained for a second cohort were measured using a second technique that is different than the first technique, all of the feature values obtained for a third cohort were measured using a third technique that is different than the first technique and the second technique, etc. More details with respect to the use of different feature measurement techniques (e.g., technical backgrounds) that are useful for method 1300 are described above with reference to method 200, and are not repeated here for brevity.

In some embodiments, e.g., some embodiments in which feature values are obtained for a plurality of cohorts of training subjects, method 1300 includes co-normalizing (1306) feature values between the first cohort and any additional cohorts. In some embodiments, feature values for features present in at least the first and second training datasets (e.g., for the first and second cohorts of training subjects) are co-normalized across at least the first and second training datasets to remove an inter-dataset batch effect, thereby calculating, for each respective training subject in the first plurality of training subjects and for each respective training subject in the second plurality of training subjects, co-normalized feature values for the plurality of modules for the respective training subject.

In some embodiments, the co-normalizing feature values present in at least the first and second training datasets (e.g., and any additional training datasets) across at least the first and second training datasets comprises estimating the inter-dataset batch effect between the first and second training datasets. In some embodiments, the inter-dataset batch effect includes an additive component and a multiplicative component and the co-normalizing solves an ordinary least-squares model for feature values across the respective first and second training datasets and shrinks resulting parameters representing the additive component and a multiplicative component using an empirical Bayes estimator. In some embodiments, the co-normalizing feature values present in at least the first and second training datasets across at least the first and second training datasets comprises making use of nonvariant features or quantile normalization.

In some embodiments, a first phenotype for a respective module in the plurality of modules represents a diseased condition, a first subset of the first training dataset consists of subjects that are free of the diseased condition, a first subset of the second training dataset (e.g., and any additional training datasets) consists of subjects that are free of the diseased condition. In some embodiments, then, the co-normalizing feature values present in at least the first and second training datasets comprises estimating the inter-dataset batch effect between the first and second training dataset using only the first subset of the respective first and second training datasets. In some embodiments, the inter-dataset batch effect includes an additive component and a multiplicative component and the co-normalizing solves an ordinary least-squares model for feature values across the first subset of the respective first and second training datasets and shrinks resulting parameters representing the additive component and a multiplicative component using an empirical Bayes estimator.

More details with respect to techniques for co-normalization across various datasets corresponding to various training cohorts that are useful for method 1300 are described above with reference to method 200, and are not repeated here for brevity.

In some embodiments, method 1300 includes summarizing (1308) feature values relating to a phenotype of the clinical condition for a plurality of modules. That is, in some embodiments, a sub-plurality of the obtained feature values (e.g., a sub-plurality of mRNA transcript abundance values) that are each associated with a particular phenotype of one or more class of the clinical condition are grouped into a module, and those grouped feature values are summarized to form a corresponding summarization of the feature values of the respective module for each training subject.

For instance, FIGS. 3 and 4 illustrate an example classifier trained to distinguish between three classes of clinical conditions, related to bacterial infection, viral infection, and neither bacterial nor viral infection. Specifically, FIG. 3 illustrates an example of a main classifier 300 that is a feed-forward neural network. Input layer 308 is configured to receive summarizations 358 of feature values 354 for a plurality of modules 352. For example, as shown on the right hand side of FIG. 4, module 352-1 includes feature values 354-1, 354-2, and 354-3, corresponding to mRNA abundance values for genes IFI27, JUP, and LAX1, that are each associated in a similar way to a phenotype of one or more of the classes of clinical conditions. In this case, IFI27, JUP, and LAX1 are all genes that are upregulated when a subject has a viral infection. As illustrated in FIG. 4, the feature values are summarized by inputting them into a feeder neural network at input layer 304, where the neural network includes a hidden layer 306 and outputs summarization 358-1, which is used as an input value for the main classifier 300. Each of the other modules 302-2 through 302-6 also include a sub-plurality of the features obtained for the subject, e.g., which is different than the sub-plurality of features in each other module, each of which are similarly associated with a different phenotype associated with one or more class of the clinical condition. For instance, the genes in module 302-2 are downregulated when a subject has a viral infection. Similarly, the genes in modules 302-3 and 302-4 are all upregulated and downregulated, respectively, in patients with sepsis as opposed to sterile inflammation. Likewise the genes in modules 302-5 and 302-6 are all upregulated and downregulated, respectively, in patients who died within 30-days of being admitted to the hospital with sepsis.

In some embodiments, method 1300 uses at least 3 modules, each of which includes features that are similarly associated with a phenotype of one or more class of a clinical condition that is evaluated by the main classifier. In some embodiments, method 1300 uses at least 6 modules, each of which includes features that are similarly associated with a phenotype of one or more class of a clinical condition that is evaluated by the main classifier. In other embodiments, method 1300 uses at least 2, 3, 4, 5, 6, 7, 8, 9, 10, 15, 20, or more modules, each of which includes features that are similarly associated with a phenotype of one or more class of a clinical condition that is evaluated by the main classifier. More details with respect to the modules, particularly with respect to grouping of features that associate with a particular phenotype that are useful for method 1300 are described above with reference to method 200, and are not repeated here for brevity.

Although the summarization method illustrated in FIG. 4 uses a feeder recurrent network, other methodologies for summarizing the features of a respective module are contemplated. Example methods for summarizing the features of a module include a neural network algorithm, a support vector machine algorithm, a decision tree algorithm, an unsupervised clustering algorithm, a supervised clustering algorithm, a logistic regression algorithm, a mixture model, or a hidden Markov model. In some embodiments, the summarization is a measure of central tendency of the feature values of the respective module. Non-limiting examples of measures of central tendency include arithmetic mean, geometric mean, weighted mean, midrange, midhinge, trimean, Winsorized mean, median, and mode of the feature values of the respective module. More details with respect to methods for summarizing feature values of a module that are useful for method 1300 are described above with reference to method 200, and are not repeated here for brevity.

Method 1300 then includes training (1310) a main classifier against (i) derivatives of the feature values from one or more cohort of training subjects and (ii) the clinical statuses of the subjects in the one or more training cohorts. In some embodiments, the main classifier is a neural network algorithm, a support vector machine algorithm, a decision tree algorithm, an unsupervised clustering algorithm, a supervised clustering algorithm, a logistic regression algorithm, a mixture model, or a hidden Markov model. In some embodiments, the main classifier is a neural network algorithm, a linear regression algorithm, a penalized linear regression algorithm, a support vector machine algorithm or a tree-based algorithm. In some embodiments, the main classifier consists of an ensemble of classifiers that is subjected to an ensemble optimization algorithm. In some embodiments, the ensemble optimization algorithm comprises adaboost, XGboost, or LightGBM. Methods for training classifiers are well known in the art. More details as to classifier types and methods for training those classifiers that are useful for method 1300 are described above with reference to method 200, and are not repeated here for brevity.

In some embodiments, the feature value derivatives are co-normalized feature values (1312). That is, in some embodiments, method 1300 includes a step of co-normalizing feature values across two or more training datasets, e.g., that are formed by feature values acquired using different measurement technologies as described above with respect to methods 200 and 1300, but not a step of summarizing groups of feature values subdivided into different modules.

In some embodiments, the feature value derivatives are summarizations of feature values (1314). That is, in some embodiments, method 1300 does not include a step of co-normalizing feature values across two or more training datasets, e.g., where a single measurement technique is used to acquire all of the feature values, but does include a step of summarizing groups of feature values subdivided into different modules, e.g., as described above with respect to methods 200 and 1300.

In some embodiments, the feature value derivatives are summarizations of co-normalized feature values (1316). That is, in some embodiments, method 1300 includes both a step of co-normalizing feature values across two or more training datasets, e.g., that are formed by feature values acquired using different measurement technologies as described above with respect to methods 200 and 1300, and a step of summarizing groups of co-normalized feature values subdivided into different modules, e.g., as described above with respect to methods 200 and 1300.

In some embodiments, the feature value derivatives are co-normalized summarizations of feature values (1318). That is, in some embodiments, method 1300 includes a first step of summarizing groups of feature values subdivided into different modules, e.g., as described above with respect to methods 200 and 1300, and a second step of co-normalizing the summarizations from the modules across two or more training datasets, e.g., that are formed by feature values acquired using different measurement technologies, using co-normalization techniques as described above with respect to methods 200 and 1300.

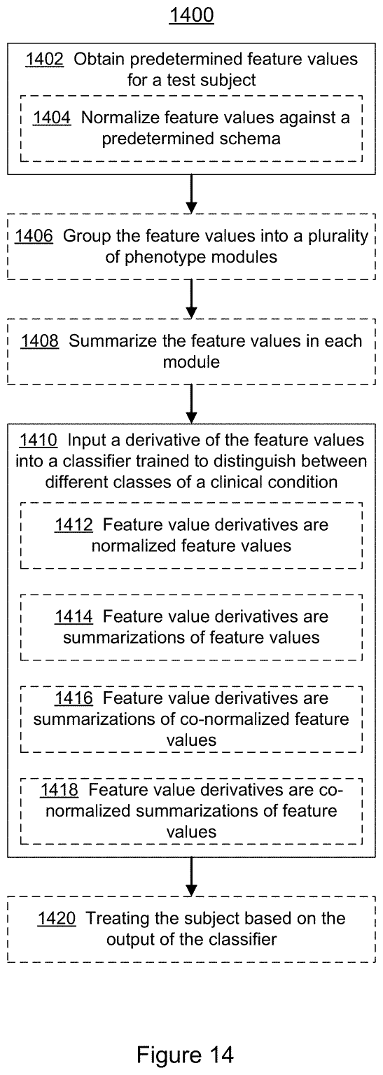

It should be understood that the particular order in which the operations in FIG. 13 are described is merely an example and is not intended to indicate that the described order is the only order in which the operations could be performed. One of ordinary skill in the art would recognize various ways to reorder the operations described herein. For example, in some embodiments, summarization (1308) of feature values for each module is performed prior to co-normalization (1306) across cohorts in which different measurement techniques were used to collect the feature data. Additionally, it should be noted that details of other processes described herein with respect to other methods described herein (e.g., method 200 described above with respect to FIG. 2 and method 1400 described below with respect to FIG. 14) are also applicable in an analogous manner to method 1300 described above with respect to FIG. 13. For example, the feature values, modules, clinical conditions, clinical phenotypes, measurement techniques, etc. described above with reference to method 1300 optionally have one or more of the characteristics of the feature values, modules, clinical conditions, clinical phenotypes, measurement techniques, etc. described herein with reference to other methods described herein (e.g., method 200 or 1400). Similarly, the methodology used at various steps, e.g., data collection, co-normalization, summarization, classifier training, etc. described above with reference to method 1300 optionally have one or more of the characteristics of the data collection, co-normalization, summarization, classifier training, etc., described herein with reference to other methods described herein (e.g., method 200 or 1400). For brevity, these details are not repeated here.

In some embodiments, the disclosure relates to a method 1400 for evaluating a clinical condition of a test subject, detailed below with reference to FIG. 14. In some embodiments, method 1400 is performed at a system as described herein, e.g., system 100 as described above with respect to FIG. 1. In some embodiments, method 1400 is performed at a system having a subset of the modules and/or databases as described with respect to system 100.

Method 1400 includes obtaining (1402) feature values for a test subject. In some embodiments, the feature values are collected from a biological sample from the test subject, e.g., as described above with respect to methods 200 and 1300 above. Non-limiting examples of biological samples include solid tissue samples and liquid samples (e.g., whole blood or blood plasma samples). More details with respect to samples that are useful for method 1400 are described above with reference to methods 200 and 1300, and are not repeated here for brevity. In some embodiments, the methods described herein include a step of measuring the various feature values. In other embodiments, the methods described herein obtain, e.g., electronically, feature values that were previously measured, e.g., as stored in one or more clinical databases.

Two examples of measurement techniques include nucleic acid sequencing (e.g., qPCR or RNAseq) and microarray measurement (e.g., using a DNA microarray, an MMChip, a protein microarray, a peptide microarray, a tissue microarray, a cellular microarray, a chemical compound microarray, an antibody microarray, a glycan array, or a reverse phase protein lysate microarray). However, the skilled artisan will know of other measurement techniques for measuring features from a biological sample. More details with respect to feature measurement techniques (e.g., technical backgrounds) that are useful for method 1400 are described above with reference to methods 200 and 1300, and are not repeated here for brevity.

In some embodiments, e.g., some embodiments in which the classifier is trained to evaluate feature values obtained from various different measurement methodologies (e.g., technical backgrounds), method 1400 includes co-normalizing (1404) feature values against a predetermined schema. In some embodiments, the predetermined schema derives from the co-normalization of feature data across two or more training datasets, e.g., that used different measurement methodologies. The various methods for co-normalizing across different training datasets are described in detail above with reference to methods 200 and 1300, and are not repeated here for brevity. In some embodiments, the feature values obtained for the test subject are not subject to a normalization that accounts for the measurement technique used to acquire the values.

In some embodiments, method 1400 includes grouping (1406) the feature values, or normalized feature values, for the subject into a plurality of modules, where each feature value in a respective module is associated in a similar fashion with a phenotype associated with one or more class of the clinical condition being evaluated. That is, in some embodiments, a sub-plurality of the obtained feature values (e.g., a sub-plurality of mRNA transcript abundance values) that are each associated with a particular phenotype of one or more class of the clinical condition are grouped into a module. In some embodiments, method 1400 uses at least 3 modules, each of which includes features that are similarly associated with a phenotype of one or more class of a clinical condition that is evaluated by the main classifier. In some embodiments, method 1400 uses at least 6 modules, each of which includes features that are similarly associated with a phenotype of one or more class of a clinical condition that is evaluated by the main classifier. In other embodiments, method 1400 uses at least 2, 3, 4, 5, 6, 7, 8, 9, 10, 15, 20, or more modules, each of which includes features that are similarly associated with a phenotype of one or more class of a clinical condition that is evaluated by the main classifier. More details with respect to the modules, particularly with respect to grouping of features that associate with a particular phenotype that are useful for method 1400 are described above with reference to methods 200 and 1300, and are not repeated here for brevity. In some embodiments, the feature values are not grouped into modules and, rather, are input directly into the main classifier.

In some embodiments, method 1400 includes summarizing (1408) the feature values in each respective module, to form a corresponding summarization of the feature values of the respective module for the test subject. For instance, as described above for module 352-1 as illustrated in FIGS. 3 and 4.

Although the summarization method illustrated in FIG. 4 uses a feeder recurrent network, other methodologies for summarizing the features of a respective module are contemplated. Example methods for summarizing the features of a module include a neural network algorithm, a support vector machine algorithm, a decision tree algorithm, an unsupervised clustering algorithm, a supervised clustering algorithm, a logistic regression algorithm, a mixture model, or a hidden Markov model. In some embodiments, the summarization is a measure of central tendency of the feature values of the respective module. Non-limiting examples of measures of central tendency include arithmetic mean, geometric mean, weighted mean, midrange, midhinge, trimean, Winsorized mean, median, and mode of the feature values of the respective module. More details with respect to methods for summarizing feature values of a module that are useful for method 1400 are described above with reference to methods 200 and 1300, and are not repeated here for brevity.

Method 1400 then includes inputting (1410) a derivative of the features values into a classifier trained to distinguish between different classes of a clinical condition. In some embodiments, the classifier is trained to distinguish between two classes of a clinical condition. In some embodiments, the classifier is trained to distinguish between at least 3 different classes of a clinical condition. In other embodiments, the classifier is trained to distinguish between at least 4, 5, 6, 7, 8, 9, 10, 15, 20, or more different classes of a clinical condition.

The main classifier is trained as described above with reference to methods 200 and 1300. Briefly, the main classifier is trained against (i) derivatives of feature values from one or more cohort of training subjects and (ii) the clinical statuses of the training subjects in the one or more training cohorts. In some embodiments, the main classifier is a neural network algorithm, a support vector machine algorithm, a decision tree algorithm, an unsupervised clustering algorithm, a supervised clustering algorithm, a logistic regression algorithm, a mixture model, or a hidden Markov model. In some embodiments, the main classifier is a neural network algorithm, a linear regression algorithm, a penalized linear regression algorithm, a support vector machine algorithm or a tree-based algorithm. In some embodiments, the main classifier consists of an ensemble of classifiers that is subjected to an ensemble optimization algorithm. In some embodiments, the ensemble optimization algorithm comprises adaboost, XGboost, or LightGBM. Methods for training classifiers are well known in the art. More details as to classifier types and methods for training those classifiers that are useful for method 1400 are described above with reference to methods 200 and 1300, and are not repeated here for brevity.

In some embodiments, the feature value derivatives are measurement platform-dependent normalized feature values (1412). That is, in some embodiments, method 1400 includes a step of normalizing the feature values based on the methodology used to acquire the feature measurements, as opposed to other measurement methodologies used in the training cohorts, as described above with respect to methods 200 and 1300, but not a step of summarizing groups of feature values subdivided into different modules.

In some embodiments, the feature value derivatives are summarizations of feature values (1414). That is, in some embodiments, method 1400 does not include a step of normalizing the feature values based on the methodology used to acquire the feature measurements, as opposed to other measurement methodologies used in the training cohorts, but does include a step of summarizing groups of feature values subdivided into different modules, e.g., as described above with respect to methods 200 and 1300.

In some embodiments, the feature value derivatives are summarizations of normalized feature values (1416). That is, in some embodiments, method 1400 includes a step of normalizing the feature values based on the methodology used to acquire the feature measurements, as opposed to other measurement methodologies used in the training cohorts, as described above with respect to methods 200 and 1300, and a step of summarizing groups of normalized feature values subdivided into different modules, e.g., as described above with respect to methods 200 and 1300.

In some embodiments, the feature value derivatives are co-normalized summarizations of feature values (1418). That is, in some embodiments, method 1400 includes a first step of summarizing groups of feature values subdivided into different modules, e.g., as described above with respect to methods 200 and 1300, and a second step of normalizing the feature values based on the methodology used to acquire the feature measurements, as opposed to other measurement methodologies used in the training cohorts, as described above with respect to methods 200 and 1300.

In some embodiments, method 1400 also includes a step of treating the test subject based on the output of the classifier. In some embodiments, the classifier provides a probability that the subject has one of a plurality of classes of the clinical condition being evaluated. When the probabilities output from the classifier positively identify one class of the clinical condition, or positively exclude a particular class of the clinical condition, treatment decision can be based on the output. For instance, where the output of the classifier indicates that the subject has a first class of the clinical condition, the subject is treated by administering a first therapy to the subject that is tailored for the first class of the clinical condition. In contrast, where the output of the classifier indicates that the subject has a second class of a clinical condition, the subject is treated by administering a second therapy to the subject that is tailored to the second class of the clinical condition.

For instance, referring to the classifier illustrated in FIG. 4, which is trained to evaluate whether a subject has a bacterial infection, has a viral infection, or has inflammation unrelated to a bacterial or viral infection. Upon input of test data to the classifier, when the classifier indicates that the subject has a bacterial infection, the subject is administered an antibacterial agent, e.g., an antibiotic. However, when the classifier indicates that the subject has a viral infection, the subject is not administered an antibiotic but may be administered an anti-viral agent. Similarly, when the classifier indicates that the subject has inflammation unrelated to a bacterial or viral infection, the subject is not administered an antibiotic or anti-viral agent, but may be administered an anti-inflammatory agent.

It should be understood that the particular order in which the operations in FIG. 14 are described is merely an example and is not intended to indicate that the described order is the only order in which the operations could be performed. One of ordinary skill in the art would recognize various ways to reorder the operations described herein. For example, in some embodiments, summarization (1408) of feature values for each module is performed prior to normalization (1404) across cohorts in which different measurement techniques were used to collect the feature data. Additionally, it should be noted that details of other processes described herein with respect to other methods described herein (e.g., method 200 described above with respect to FIG. 2 and method 1300 described above with respect to FIG. 13) are also applicable in an analogous manner to method 1400 described above with respect to FIG. 14. For example, the feature values, modules, clinical conditions, clinical phenotypes, measurement techniques, etc. described above with reference to method 1400 optionally have one or more of the characteristics of the feature values, modules, clinical conditions, clinical phenotypes, measurement techniques, etc. described herein with reference to other methods described herein (e.g., method 200 or 1300). Similarly, the methodology used at various steps, e.g., data collection, co-normalization, summarization, classifier training, etc. described above with reference to method 1400 optionally have one or more of the characteristics of the data collection, co-normalization, summarization, classifier training, etc., described herein with reference to other methods described herein (e.g., method 200 or 1300). For brevity, these details are not repeated here.

### Example 1

### Systematic Search and Inclusion Criteria for Gene Expression Studies of Clinical Infection

IMX training datasets for studies of clinical infections matching defined inclusion criteria were obtained from the NCBI GEO (www.ncbi.nlm.nih.gov/geo/) and EMBL-EBI ArrayExpress (www.ebi.ac.uk/arrayexpress) databases. Specifically, the inclusion criteria included that patients in the study 1) had to be physician-adjudicated for the presence and type of infection (e.g. strictly bacterial infection, strictly viral infection, or non-infected inflammation), 2) had gene expression measurements of the 29 diagnostic markers identified previously by Sweeney et al. (Sweeney et al., 2015, Sci Transl Med 7(287), pp. 287ra71; Sweeney et al, 2016, Sci Transl Med 8(346), pp. 346ra91; and Sweeney et al., 2018, Nature Communications 9, p. 694), 3) were over 18 years of age, 4) had been seen in hospital settings (e.g. emergency department, intensive care), 5) had either community- or hospital-acquired infection, and 6) had blood samples taken within 24 hours of initial suspicion of infection and/or sepsis. In addition, the normalization/batch effect control approach used required that each included study must have assayed at least control samples (e.g., samples not diagnosed with any of the three conditions under consideration). Studies in which patients experienced trauma or had conditions either not encountered in a typical clinical setting (e.g. experimental LPS challenge) or confused with infection (e.g. anaphylactic shock) were excluded.

### Example 2

### Normalization and COCONUT Co-Normalization of Expression Data

Normalization was then performed within each study, adopting one of two approaches depending on the platform. For Affymetrix arrays, the expression data was normalized using either Robust Multi-array Average (RMA) (Irizarry et al., 2003, Biostatistics, 4(2):249-64) or gcRMA (Wu et al., 2004, Journal of the American Statistical Association, 99:909-17). Expression data from other platforms were normalized using an exponential convolution approach for background correction followed by quantile normalization.

Following normalization of the raw expression data, the COCONUT algorithm (Sweeney et al., 2016, Sci Transl Med 8(346), pp. 346ra91; and Abouelhoda et al., 2008, BMC Bioinformatics 9, p. 476) was used to co-normalize these measurements and ensure that they were comparable across studies. COCONUT builds on the ComBat (Johnson et al., 2007, Biostatistics, 8, pp. 118-127) empirical Bayes batch correction method, computing the expected expression value of each gene from healthy patients and adjusting for study-specific modifications of location (mean) and scale (standard deviation) in the gene's expression. For this analysis, the parametric prior of ComBat in which gene expression distributions are assumed to be Gaussian and the empirical prior distributions for study-specific location and variance modification parameters are Gaussian and Inverse-Gamma, respectively, were used.

### Example 3

### Sepsis Classifier Development by Machine Teaming

To develop a classifier for sepsis, a machine learning approach was employed. The approach included specifying candidate models, assessing the performance of different classifiers using training data and a specified performance statistic, and then selecting the best performing model for evaluation on independent data.

In this context, the model refers to a machine learning algorithm, such as logistic regression, neural network, decision tree, etc., similar to models used in statistics. Similarly, in this context, a classifier refers to a model with fixed (locked) parameters (weights) and thresholds, ready to be applied to previously unseen samples. Classifiers use two types of parameters: weights, which are learned by the core learning algorithm (such as XGBoost), and additional, user-supplied parameters which are inputs to the core learner. These additional parameters are referred to as hyperparameters. Classifier development entails learning (fixing) weights and hyperparameters. The weights are learned by the core learning algorithms; to learn hyperparameters. For this study, a random search methodology was employed (Bergstra et al., 2012, Journal of Machine Learning Research 13, pp. 281-305).

The performance of four different types of predictive models: 1) logistic regression with a lasso (L1) penalty, 2) support vector machine (SVM) classifiers with radial basis function kernels (RBF), 3) extreme gradient-boosted trees (XGBoost), and 4) multi-layer perceptrons (MLPs) were compared. Each type of predictive model was evaluated for its accuracy in classifying patient samples as one of: a) strictly bacterial infection, b) strictly viral infection, or c) non-infected inflammation.

To evaluate each predictive model on this three-class classification task, a metric called average pairwise area-under-the-ROC curve (APA) was developed. APA is defined as the average of the three one-class-versus-all (OVA) areas-under-the-ROC curve; that is, the average of bacterial-vs-other AUC, viral-vs-other AUC, and noninfected-vs-other AUC.

A variety of approaches for assessing performance of a particular classifier (e.g., a model with a fixed set of weights and hyperparameters) can be used in machine learning. Here, cross-validation (CV), a well-established method for small sample scenarios such as sepsis research, was employed. Two CV variants were used, described below.

### Example 4

### Model Cross-Validation Approaches

Two different types of CV schemes were initially considered: conventional 5-fold cross-validation and leave-one-study-out (LOSO) cross-validation. For trials of 5-fold CV, standard methodology for randomly partitioning all IMX samples into five non-overlapping subsets of roughly similar sample sizes was used. For trials of LOSO CV, each study was treated as a CV partition. In this way, at each step (“fold”) in LOSO CV, a candidate model is trained on all studies but one, and the trained model is then used to generate predictions for the remaining study.

The rationale for using LOSO CV is as follows. Briefly, an assumption of k-fold CV is that the cross-validation training and validation samples are drawn from the same distribution. However, due to extraordinary heterogeneity of sepsis studies, this assumption is not even approximately satisfied. LOSO is designed to favor models which are, empirically, the most robust with respect to this heterogeneity; in other words, models which are most likely to generalize well to previously unseen studies. This is a critical requirement for clinical application of sepsis classifiers.

The LOSO method is related to prior work which proposed clustering of training data prior to cross-validation as a means of accounting heterogeneity (Tabe-Bordbar, 2018, et al., Sci Rep 8(1), pp. 6620). In this case, clustering is not needed because the clusters naturally follow from the partitioning of the training data to studies.

In both k-fold CV and LOSO, the predictions were pooled in the left-out folds across all folds to evaluate model performance. Alternatively, it is possible to compute CV statistics by estimating statistics of interest on each fold, and then averaging the per-fold results. In the present study, LOSO requires pooling because the majority of studies do not have samples from all three classes, and therefore most statistics of interest are not computable on individual LOSO folds. Given this situation, and for fair comparison with k-fold CV, the pooling method was applied uniformly.

To determine appropriate cross-validation schemes and feature sets for the selection and prospective validation of the diagnostic classifier, hierarchical cross-validation (HCV) was used. HCV is technically equivalent to nested CV (NCV). However, it is referred to as HCV here because it is used for a different purpose than NCV. Specifically, in NCV, the goal is estimating performance of an already selected model. In contrast, HCV is used here to evaluate and compare components (steps) of the model selection process.

HCV partitions IMX dataset into three folds; each fold is constructed such that all samples from a given study only appear in one fold. These three HCV folds were manually constructed to have similar compositions of bacterial, viral and non-infected samples. To evaluate 5-fold and LOSO CV in this framework, each CV approach was performed on the samples from two of the HCV folds (the inner fold). The models were then ranked by their CV performance (in terms of APA) on the inner fold, and evaluated the top 100 models from each CV approach on the remaining third HCV fold (the outer fold). This procedure was carried out three times, each time setting the outer fold to one HCV fold and the inner fold to the remaining two HCV folds.

### Example 5

### Predictive Model Evaluation and Hyperparameter Search

Uncovering promising candidate predictive models involves identifying values of each model's hyperparameters that lead to robust generalization performance. The four predictive models evaluated here can be broadly categorized as models with small (low-dimensional) or large (high-dimensional) numbers of hyperparameters. More specifically, the predictive models with low-dimensional hyperparameter spaces are logistic regression with a lasso penalty and SVM while the predictive models with high-dimensional hyperparameter spaces are XGBoost and MLP. For predictive models with low-dimensional hyperparameter spaces, 5000 model instances (different values of the model's corresponding hyperparameters) were sampled for evaluation in cross-validation. For predictive models with high-dimensional hyperparameter spaces (e.g. xgboost and MLP), 100,000 model instances were randomly sampled. In the case of logistic regression, there is only one hyperparameter to consider: the lasso penalty coefficient. For SVM, values of the C penalty term and the kernel coefficient, gamma, were sampled. For XGBoost, the following hyperparameters were sampled: 1) the pseudo-random-number generator seed, 2) the learning rate, 3) the minimum loss reduction required to introduce a split in the classifier tree, 4) the maximum tree depth, 5) the minimum child weight, 6) the minimum sum of instance weights required in each child, 7) the maximum delta step, 8) the L2 penalty coefficient for weight regularization, 9) the tree method (exact or approximate), and 10) the number of rounds. For MLP, the batch size was fixed to 128 and the optimization algorithm to ADAM. The following hyperparameters were then sampled: 1) the number of hidden layers, 2) the number of nodes per hidden layer, 3) the type of activation function for each hidden layer (e.g. ReLU and variants, linear, sigmoid, tan h), 4) the learning rate, 5) the number of training iterations, 6) the type of weight regularization (L1, L2, none), and 7) the presence (whether to enable or not) and amount (probabilities) of dropout for the input and hidden layers. The number of nodes per hidden layer is the same across all hidden layers. The β1, β2, and ε parameters of ADAM were fixed to 0.9, 0.999, and 1e-08, respectively.

In the cases of both XGBoost and MLP, some hyperparameters were sampled uniformly from a grid and others from continuous ranges following the approach by Bergstra & Bengio, supra.

### Example 6

### Fine-Tuning of Neural Network Hyperparameters

In the neural network analyses, observed significant variation of results was observed with respect to the seed value used to initialize the network weights. To account for this variability, multiple methods were considered, including a variety of ensemble models. Based on empirical evidence, an approach of including the seed as an additional hyperparameter in the search was adopted. The “core” hyperparameters were searched randomly, whereas seed was searched exhaustively, using a fixed pre-defined list of 1000 values.

The addition of the random seed significantly increased the hyperparameter search space. To reduce the amount of computations, a with large grid of hyperparameters (except seed) were used as a starting poing. For each random sample from the grid, over 250 seed values were searched. Upon completion of the initial search, a smaller grid of most promising hyperparameters were selected. The hyperparameter values were then refined by searching in the vicinity of the promising hyperparameter configurations. For each randomly sampled fine-tuning point, an additional larger set of seed values (e.g., 750) was searched. The configuration with the largest APA was selected as the final, locked set of hyperparameter values. This set included the random number generator seed.

### Example 7

### Diagnostic Marker and Geometric Mean Feature Sets

Two sets of input features were considered in these analyses. The first set consists of 29 gene markers previously identified as being highly discriminative of the presence, type and severity of infection (Sweeney et al., 2015, Sci Transl Med 7(287), pp. 287ra71; Sweeney et al, 2016, Sci Transl Med 8(346), pp. 346ra91; and Sweeney et al., 2018, Nature Communications 9, p. 694). The second set of input features was based on modules (subsets of related genes). The 29 genes were split in 6 modules such that each module consists of genes which share expression pattern (trend) in a given infection or severity condition. For example, genes in the fever-up module are overexpressed (up-regulated) in patients with fever. The composition of the modules is shown in Table 1.

TABLE 1
Definition and composition of sepsis-related modules (sets of genes).
Fever-up/down: genes with elevated/reduced
expression in strictly viral infection. Sepsis-up/down:
genes with elevated/reduced expression in patients with sepsis vs.
sterile inflammation. Severity-up/down: genes with elevated/
reduced expression in patients who
died within 30 days of hospital admission.

| MODULE | GENES |
|---|---|
| Fever-up | IFI27, JUP, LAX1 |
| Fever-down | HK3, TNIP1, GPAA1, CTSB |
| Sepsis-up | CEACAM1, ZDHHC19, C9orf95, GNA15, |
| | BATF, C3AR1 |
| Sepsis-down | KIAA1370, TGFBI, MTCH1, RPGRIP1, HLA- |
| | DPB1 |
| Severity-up | DEFA4, CD163, RGS1, PER1, HIF1A, |
| | SEPP1, C11orf74, CIT |
| Severity-down | LY86, TST, KCNJ2 |

The module-based features used in these analyses are the geometric means computed from the expression values of genes in each module, resulting in six geometric mean scores per patient sample. This approach may be viewed as a form of “feature engineering,” a method known to sometimes significantly improve machine learning classifier performance.

### Example 8

### Alignment of IMX and ICU Datasets by Iterative Application of COCONUT

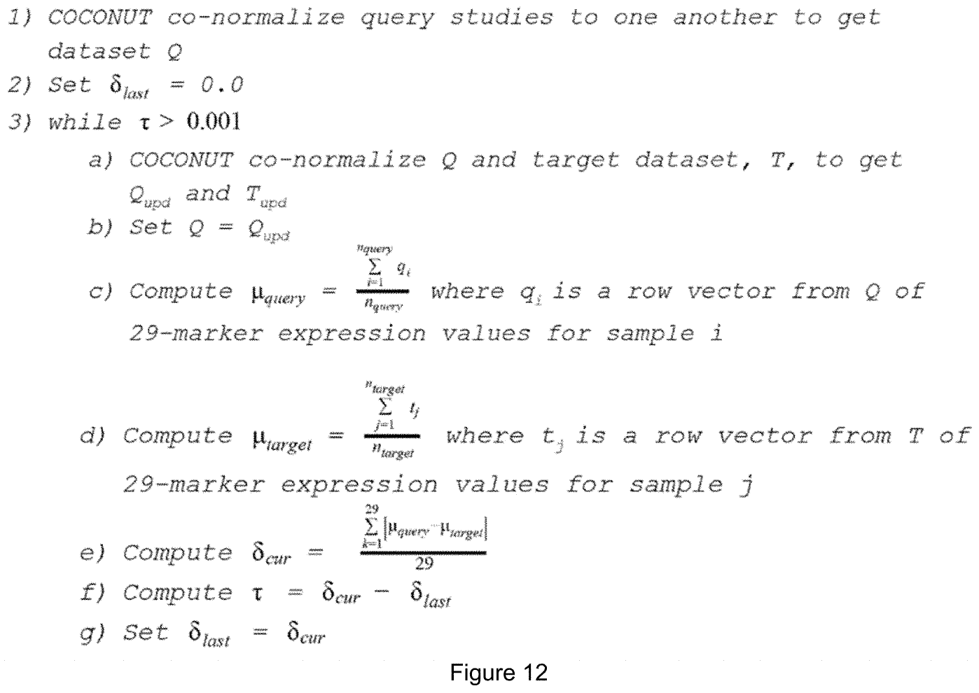

Externally validating predictive models trained on IMX with the validation clinical dataset required first making expression levels comparable across the different technical platforms (e.g., microarray for IMX and NanoString for validation clinical data) used to generate the two datasets. Following normalization of the raw expression data, we used the COCONUT algorithm (Sweeney et al., 2016, Sci Transl Med 8(346), pp. 346ra91) to co-normalize these measurements and ensure that they were comparable across studies. COCONUT builds on the ComBat (Johnson et al., 2007, Biostatistics, 8, pp. 118-127) empirical Bayes batch correction method, computing the expected expression value of each gene from healthy patients and adjusting for study-specific modifications of location (mean) and scale (standard deviation) in the gene's expression. For this analyses, we used the parametric prior of ComBat in which gene expression distributions are assumed to be Gaussian and the empirical prior distributions for study-specific location and variance modification parameters are Gaussian and Inverse-Gamma, respectively. Advantageously, the COCONUT algorithm was applied iteratively, applying co-normalization to the healthy samples of the IMX dataset while keeping the healthy samples of the validation clinical dataset unmodified at each step. In this setting, the NanoString healthy samples represent the target dataset as it remains unchanged over the course of the procedure and the IMX healthy samples represent the query dataset that is being made similar to the target dataset. This procedure terminated when the mean absolute deviation (MAD) between the vectors of average expression of the 29 diagnostic markers in both IMX and NanotString did not change by more than 0.001 in consecutive iterations. More detailed pseudocode for the procedure appears in FIG. 12.

In accordance with FIGS. 1 and 12, the present disclosure provides a computer system 100 for dataset co-normalization, the computer system comprising at least one processor 102 and a memory 111/112 storing at least one program (e.g., data co-normalization module 124) for execution by the at least one processor.

The at least one program further comprises instructions for (A) obtaining in electronic form a first training dataset. The first training dataset comprises, for each respective training subject in a first plurality of training subjects of the species: (i) a first plurality of feature values, acquired using a biological sample of the respective training subject, for a plurality of features and (ii) an indication of the absence, presence or stage of a clinical condition in the respective training subject, and wherein a first subset of the first training dataset consists of subjects do not exhibit the clinical condition (e.g., the Q dataset of FIG. 12).

The at least one program further comprises instructions for (B) obtaining in electronic form a second training dataset. The second training dataset comprises, for each respective training subject in a second plurality of training subjects of the species: (i) a second plurality of feature values, acquired using a biological sample of the respective training subject, for the plurality of features and (ii) an indication of the absence, presence or stage of the clinical condition in the respective training subject and wherein a first subset of the second training dataset consists of subjects that do not exhibit the clinical condition (e.g., the T dataset of FIG. 12).

The at least one program further comprises instructions for (C) estimating an initial mean absolute deviation between (i) a vector of average expression of the subset of the plurality of features across the first plurality of subjects and (ii) a vector of average expression of the subset of the plurality of features across the second plurality of subjects (e.g., FIG. 12, step 2). For instance, as set forth in FIG. 12, step 2, in some embodiments the estimating the initial mean absolute deviation (C) between (i) a vector of average expression of the subset of the plurality of features across the first plurality of subjects and (ii) a vector of average expression of the subset of the plurality of features across the second plurality of subjects comprises setting the initial mean absolute deviation to zero.

The at least one program further comprises instructions for (D) co-normalizing feature values for a subset of the plurality of features across at least the first and second training datasets to remove an inter-dataset batch effect, where the subset of features is present in at least the first and second training datasets, the co-normalizing comprises estimating an inter-dataset batch effect between the first and second training dataset using only the first subset of the respective first and second training datasets, and the inter-dataset batch effect includes an additive component and a multiplicative component and the co-normalizing solves an ordinary least-squares model for feature values across the first subset of the respective first and second training datasets and shrinks resulting parameters representing the additive component and the multiplicative component using an empirical Bayes estimator, thereby calculating using the resulting parameters: for each respective training subject in the first plurality of training subjects, co-normalized feature values of each feature value in the plurality of features (e.g., FIG. 12, step 3a and as disclosed in Sweeney et al., 2016, Sci Transl Med 8(346), pp. 346ra91).

The at least one program further comprises instructions for (F) estimating a post co-normalization mean absolute deviation between (i) a vector of average expression of the co-normalized feature values of the plurality of features across the first training dataset and (ii) a vector of average expression of the subset of the plurality of features across the second training dataset (e.g., FIG. 12, steps 3b, 3c, 3d, and 3e).

The at least one program further comprises instructions for (G) repeating the co-normalizing (E) and the estimating (F) until the co-normalization mean absolute deviation converges (e.g., FIG. 12, step 3f and 3g and the while condition τ>0001 of step 3).

### Example 9

### Commercial Healthy Samples for General Alignment to NanoString Expression Data

Deployment of the above iterative COCONUT procedure in clinical settings would be infeasible, since it would require acquisition of healthy samples at the site of deployment and realignment of all healthy samples (both previously and newly acquired). To establish a general model of NanoString expression in healthy patients, a set of 40 commercially available healthy control samples with ten PAXGENE™ whole blood RNA samples, each acquired from four different sites in the continental USA, was identified. Donors that provided these samples self-reported as healthy and received negative test results for both HIV and hepatitis C. In terms of gender, 12 of the healthy samples were from female donors while the remaining 28 samples were taken from male donors.

### Example 10

### Validation Clinical Study Sample Description and NanoString Expression Profiling

Patients admitted to a hospital for suspected sepsis were enrolled for this study. To generate NanoString expression for the ICU samples, RNA was isolated with the RNeasy Plus Micro Kit (Qiagen, part #74034) on a QIAcube (Qiagen), following extraction of PAXgene RNA for each sample, using a custom script for the QIAcube for RNA isolation. Each expression profiling reaction consisted of 150 ng of RNA per sample. A custom code set of probes to detect expression of our biomarker panel, and sample RNA was hybridized for 16 hours at 65° C. per manufacturer's instructions. The nCounter SPRINT standard protocol was then used to generate NanoString expression which resulted in raw RCC expression files. No normalization was performed on these raw expression values. Following the processing, a total of 104 data samples were available for analyses.

As described above, 18 studies were identified in public domain which met inclusion criteria and were used for classifier training. The studies comprised 1069 distinct patient samples. The composition and key characteristics of the studies are shown in Table 2.

TABLE 2
Characteristics of training studies. ED = Emergency Department; ICU =
Intensive Care Unit. ED/ICU is number (percentage) of samples collected in ED (the rest
were from ICU). Platform = gene expression platform. Numbers in parentheses indicate
percentages.

| STUDY | N | BAC. | | VIR. | | NON-INF. | | MALE | | FEM. | | UNK. | | ED/ICU | | P¹ |
|---|---|---|---|---|---|---|---|---|---|---|---|---|---|---|---|---|
| A | 23 | 4 | (17) | 5 | (22) | 14 | (61) | 5 | (22) | 16 | (70) | 2 | (9) | 10 | (43) | A |
| B | 140 | 82 | (59) | | | 58 | (41) | 44 | (31) | 95 | (68) | 1 | (1) | 140 | (100) | A |
| C | 228 | 228 | (100) | | | | | 100 | (44) | 128 | (56) | | | 228 | (100) | I |
| D | 33 | | | 33 | (100) | | | 18 | (55) | 15 | (45) | | | 0 | (0) | I |
| E | 45 | 45 | (100) | | | | | 19 | (42) | 26 | (58) | | | | | I |
| F | 15 | 15 | (100) | | | | | 9 | (60) | 6 | (40) | | | | | I |
| G | 10 | 6 | (60) | 4 | (40) | | | 6 | (60) | 4 | (40) | | | | | I |
| H | 12 | | | 12 | (100) | | | | | | | 12 | (100) | 12 | (100) | I |
| I | 7 | | | 7 | (100) | | | 1 | (14) | 6 | (86) | | | 7 | (100) | A |
| J | 21 | 10 | (48) | | | 11 | (52) | | | | | 21 | (100) | | | A |
| K | 34 | 16 | (47) | 6 | (18) | 12 | (35) | 15 | (44) | 19 | (56) | | | 34 | (100) | I |
| L | 82 | 14 | (17) | | | 68 | (83) | 35 | (43) | 32 | (39) | 15 | (18) | 0 | (0) | I |
| M | 82 | 82 | (100) | | | | | 27 | (33) | 55 | (67) | | | 82 | (100) | A |
| N | 93 | 22 | (24) | 71 | (76) | | | 56 | (60) | 37 | (40) | | | 0 | (0) | I |
| O | 33 | | | | | 33 | (100) | 11 | (33) | 22 | (67) | | | 33 | (100) | A |
| P | 104 | | | 104 | (100) | | | 54 | (52) | 50 | (48) | | | 0 | (0) | I |
| Q | 83 | 83 | (100) | | | | | | | | | 83 | (100) | | | I |
| R | 24 | | | 24 | (100) | | | 10 | (42) | 14 | (58) | | | 0 | (0) | A |

¹Platform: A = Agilent, I = Illumina

Normalization

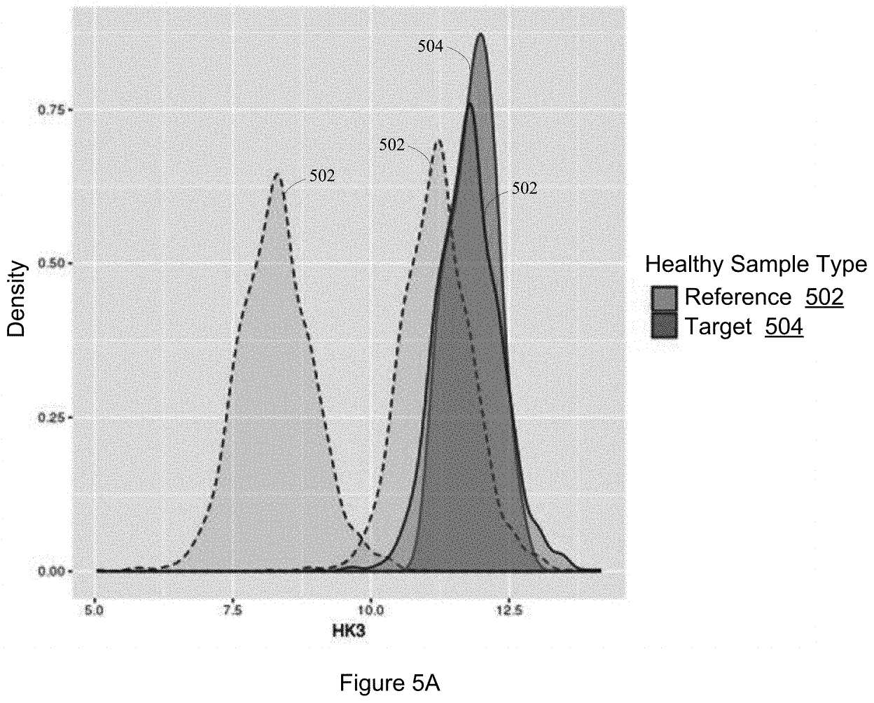

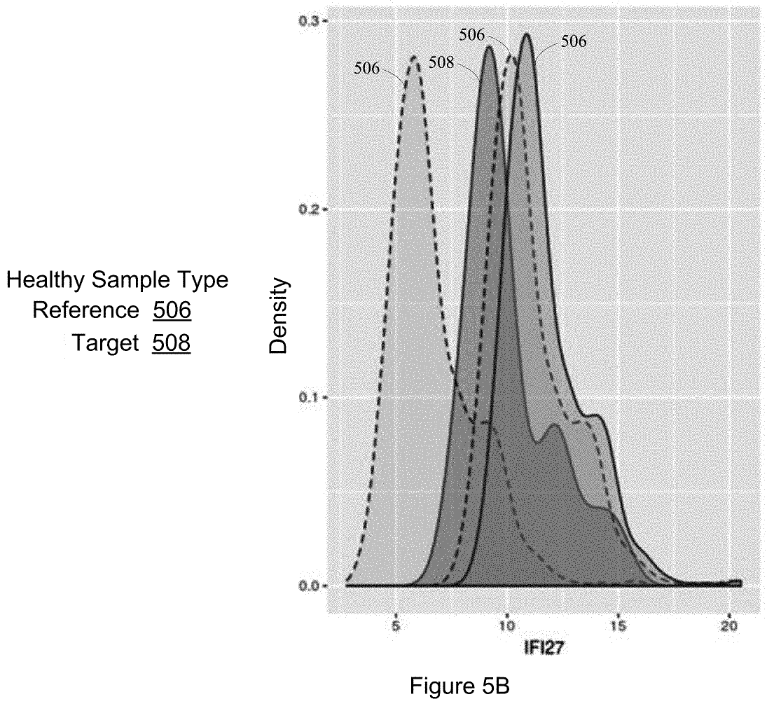

According to procedure described above, study-normalized training data were iteratively adjusted using COCONUT, PROMPT data and the 40 commercial control samples processed on NanoString instrument. The resulting batch-adjusted training data entered into exploratory data analyses and machine learning. To illustrate the iterative process of COCONUT co-normalization, plotted distribution of selected genes in the training set before, during and following the normalization is plotted in FIG. 5. The distributions in the target and query datasets become visually closer with iterations, as expected.

Exploratory Data Analysis

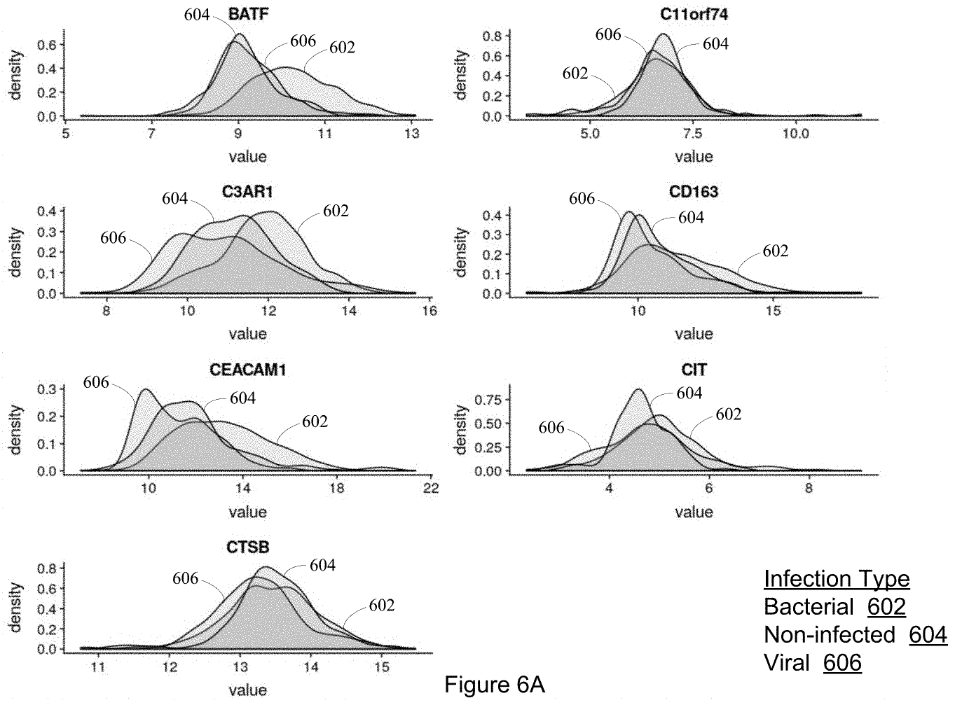

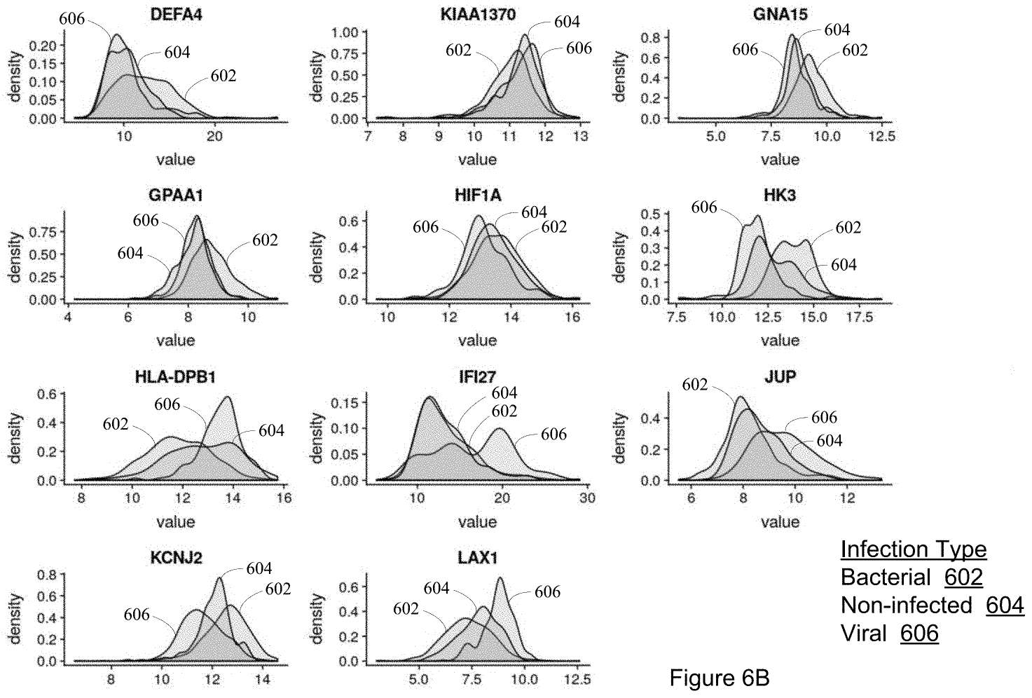

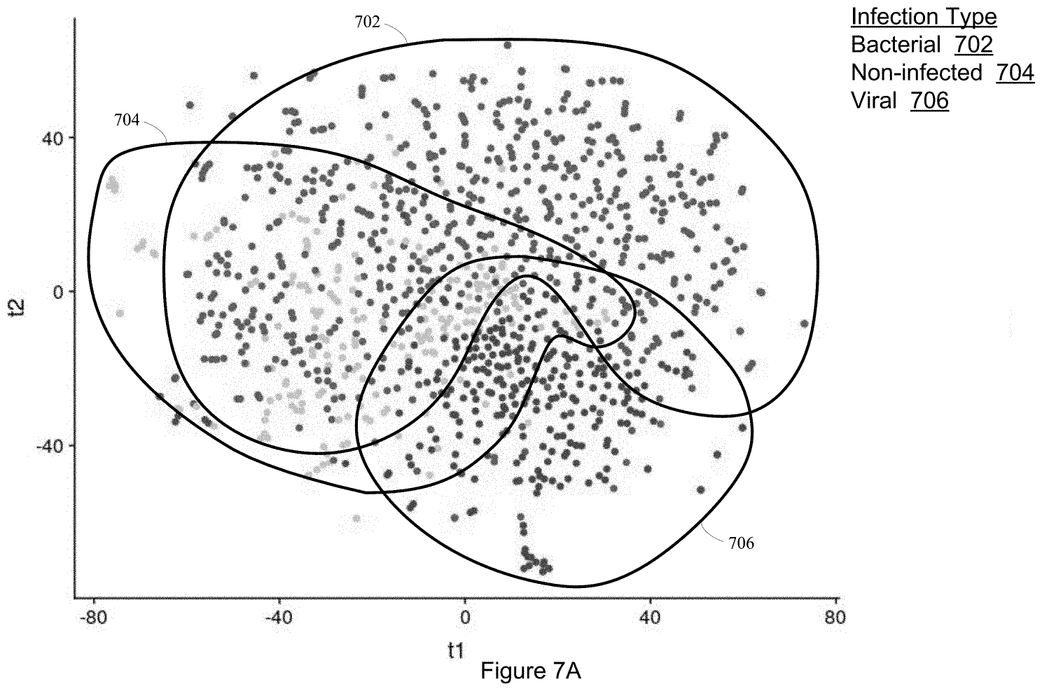

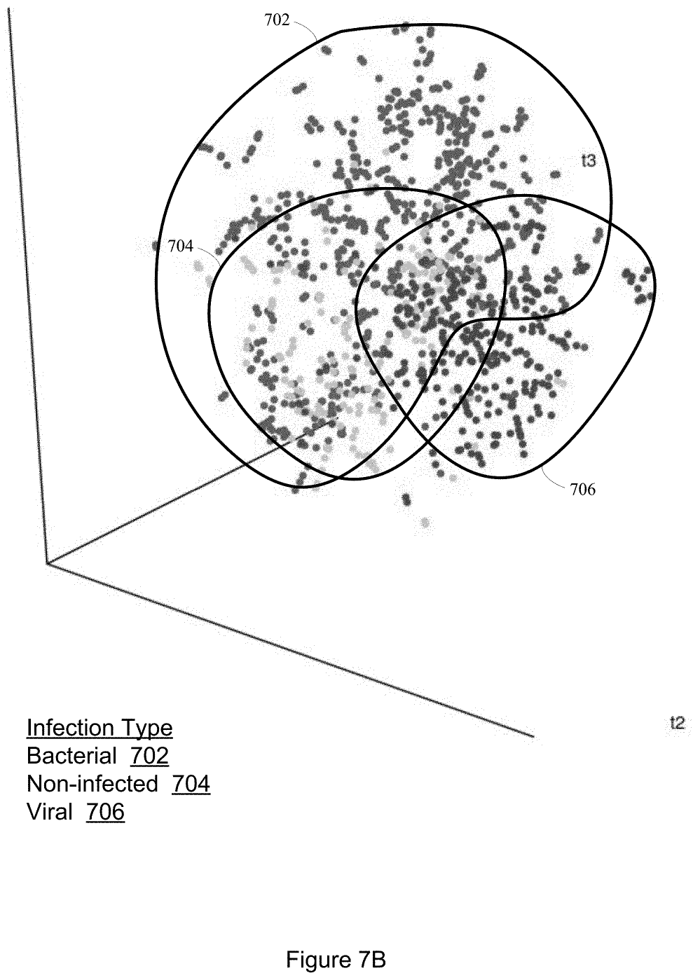

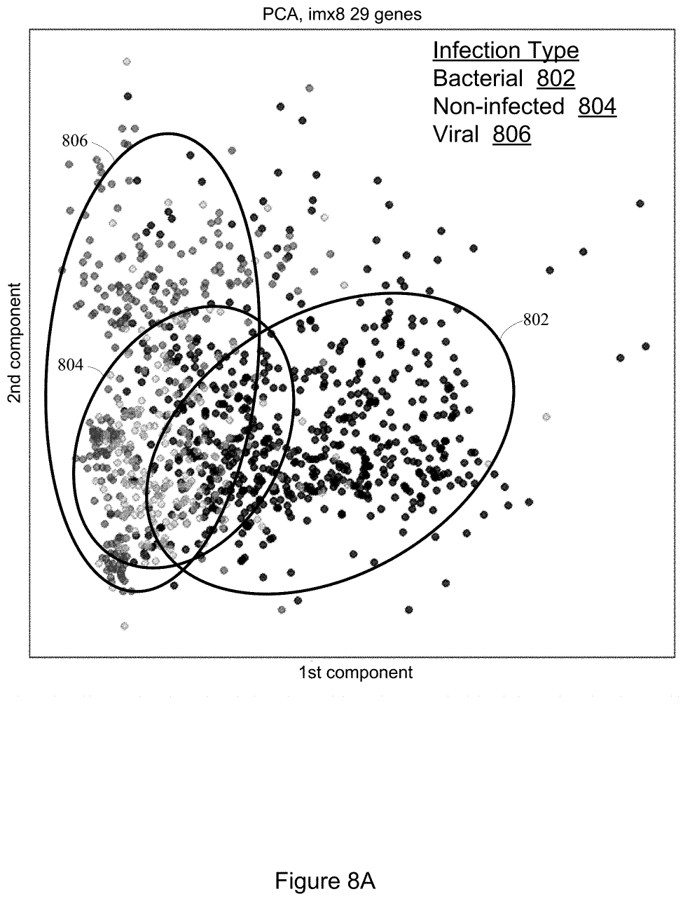

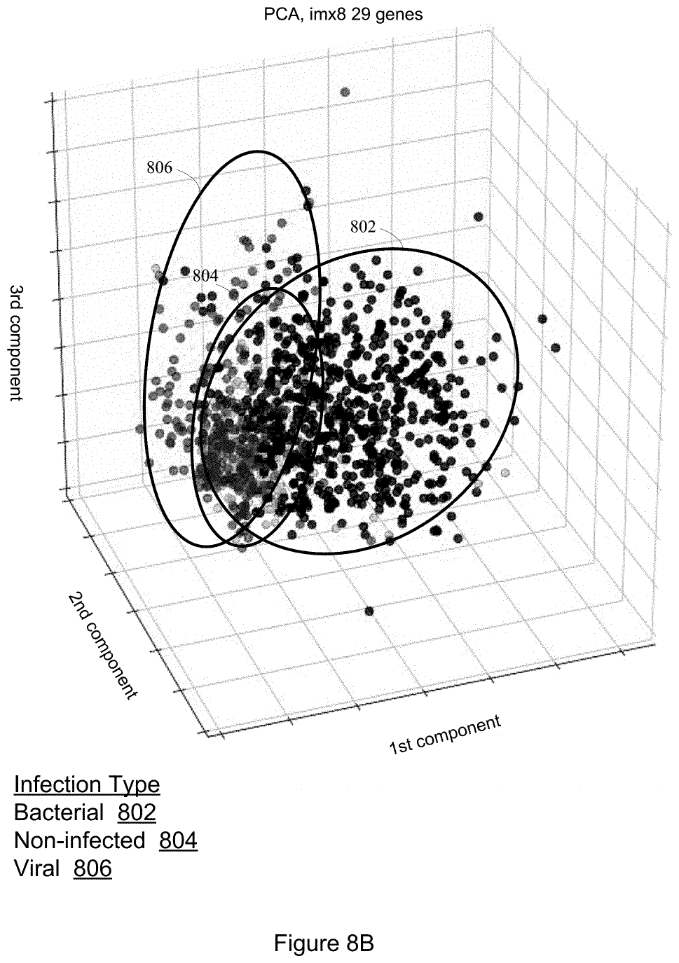

The distributions of co-normalized expression values of bacterial, viral and non-infected samples for each of the 29 genes used in the algorithm were then visualized, as shown in FIG. 6. The histograms suggested modest (bacterial vs. viral) to minimal (non-infected) separation of the classes at the individual gene level, and the need for advanced multi-gene modeling in order to achieve clinical utility of the sepsis classifier. Next, projection of the three-class data was visualized to 2 and 3 dimensions using t-distributed stochastic neighbor embedding (t-SNE), as shown in FIG. 7, and Principal Component Analysis (PCA), as shown in FIGS. 8A and 8B. Both analyses confirmed the initial finding of needing to develop high-dimensional classifier to reach clinically viable performance.

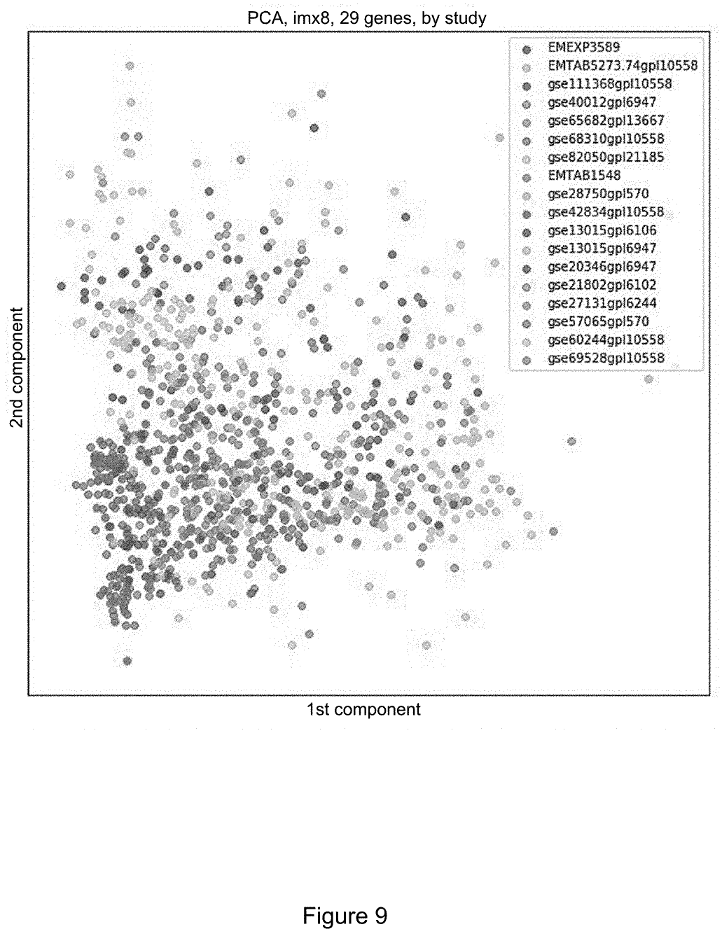

The samples were also plotted by study in the two-dimensional PCA space, as shown in FIG. 9. This result suggested that there was a residual study effect following normalization by COCONUT. This observation, along with prior research in the field, suggested that classifiers must be tested on distinct, previously unseen studies, to avoid confounding by the study (e.g., to avoid learning a batch instead of the disease signal). This is particularly important given that some studies in the training set were single-disease.

Leave-One-Study-Out Vs. Cross-Validation

The disease heterogeneity and the residual batch effect suggested that ordinary cross-validation for model selection may be subject to significant overfitting. To test this hypothesis, comparative analysis of two model selection methods were performed: 5-fold cross-validation and leave-one-study-out cross-validation. The analysis used 3-fold hierarchical cross-validation (HCV), in which each outer fold simulates an independent validation of the best classifier selected in the inner loop. This exposes potential overfitting of a particular classifier selection method without the need for a separate (and unavailable) validation set. The studies were combined such that the class distributions in each partition were as similar as possible.

In HCV, each inner loop performed classifier tuning, using either standard CV or LOSO. To select the best model, we ranked candidates by Average Pairwise AUROC statistic (APA). The reasons for choosing APA were: (1) in preliminary analyses it showed most concordant behavior between training and test data of all relevant statistics, (2) it is clinically highly relevant in diagnosing sepsis, and (3) the choice of the model selection statistic was not considered critical because prior evidence suggested that the gap between generalization ability of CV and LOSO was substantial. In other words, other statistics could have been used, but APA was a straightforward choice.

The comparison was performed using the SVM with RBF kernel, deep learning MLP, logistic regression (LR) and XGBoost classifiers. The rationale for using these classifiers was: (1) for SVM, prior experience, use in existing clinical diagnostic tests, (2) for LR, the wide acceptance in medicine in general, and diagnosis of infectious disease in particular, (3) for XGBoost, the wide acceptance in machine learning community and track record of top performance in major competitive challenges, such as Kaggle, and (4) for deep neural networks, the recent breakthrough results in multiple application domains (image analysis, speech recognition, Natural Language Processing, reinforcement learning).

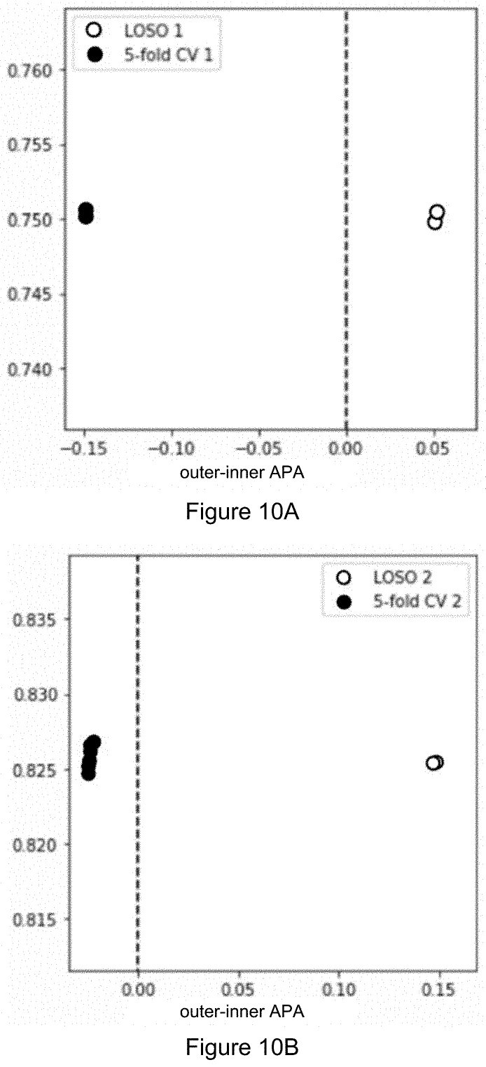

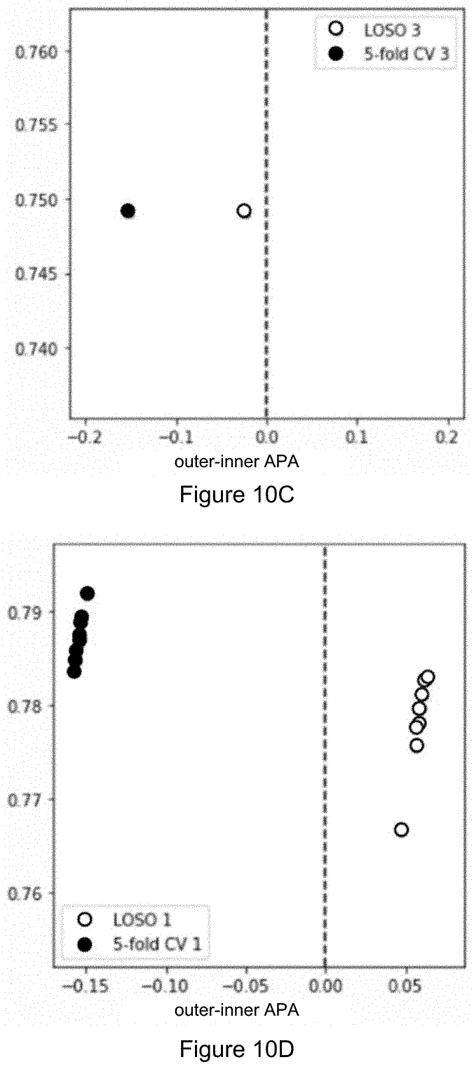

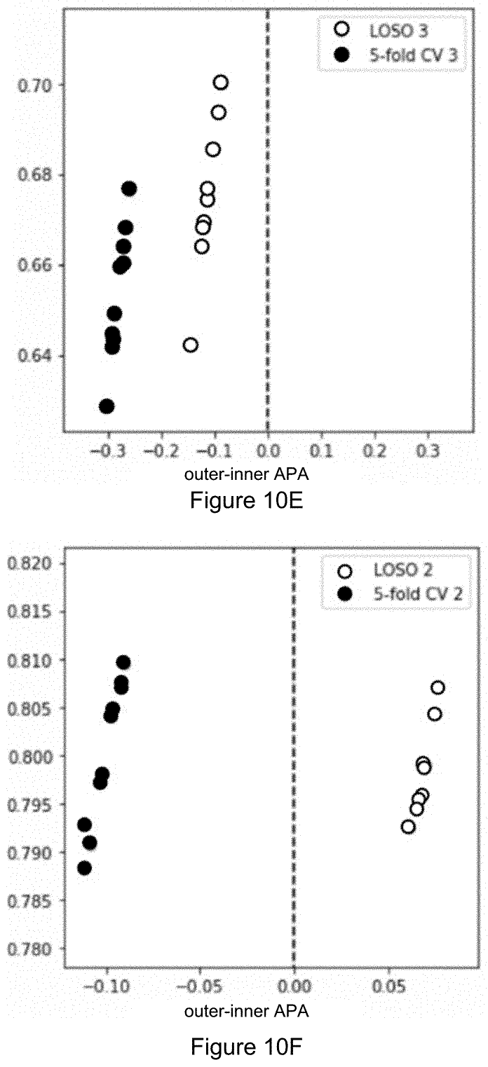

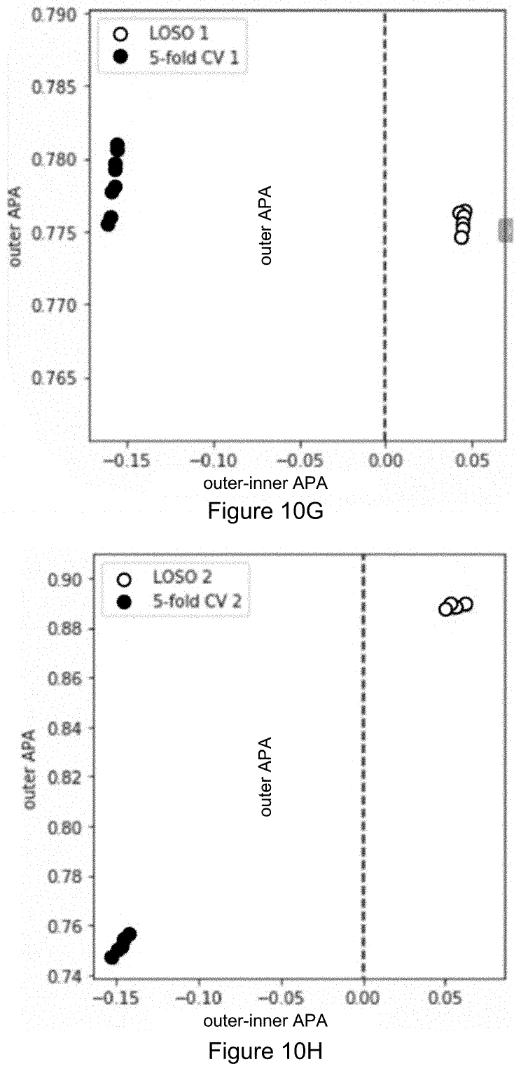

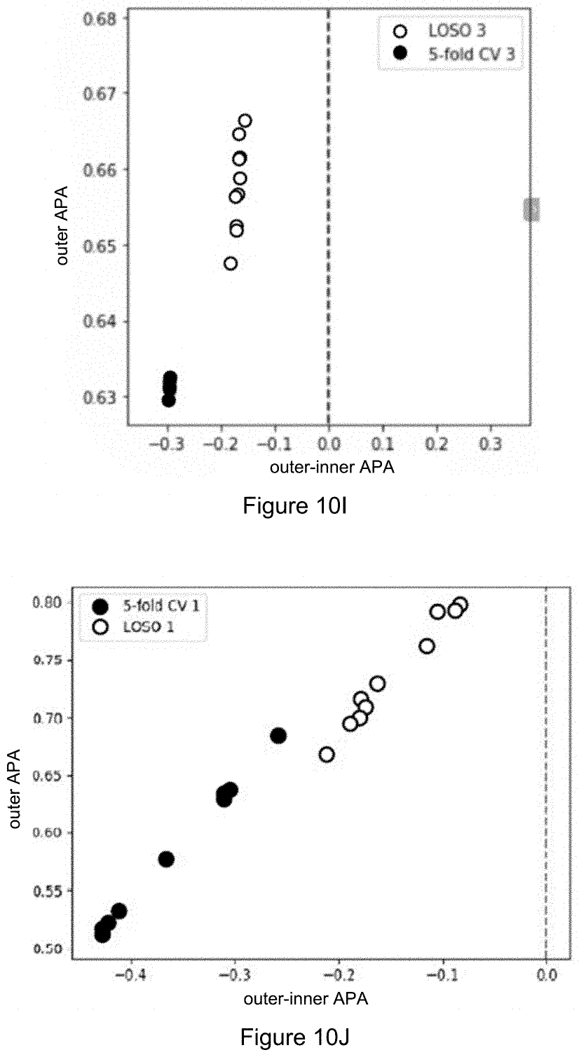

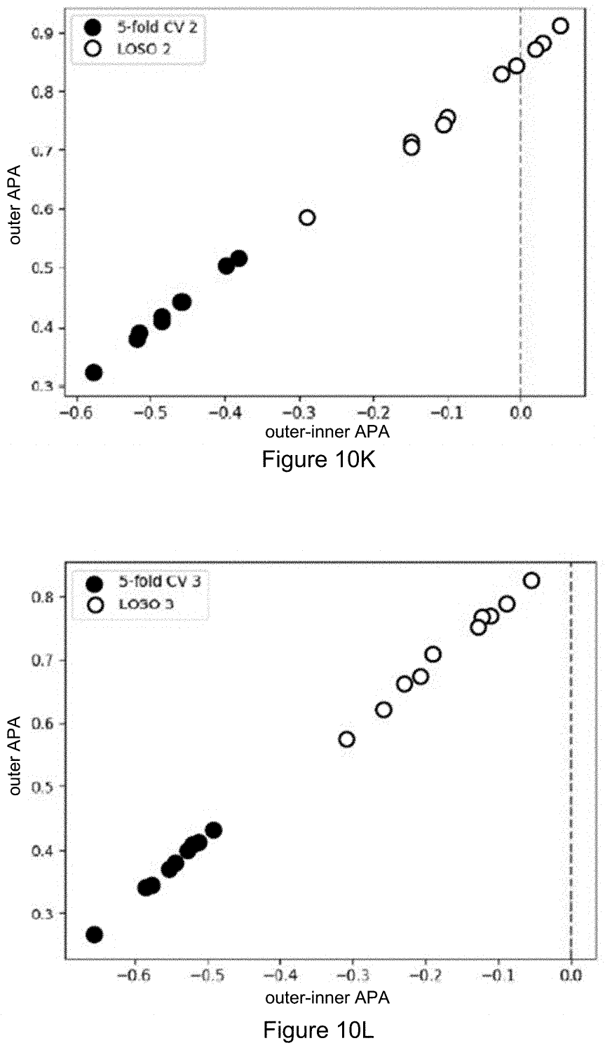

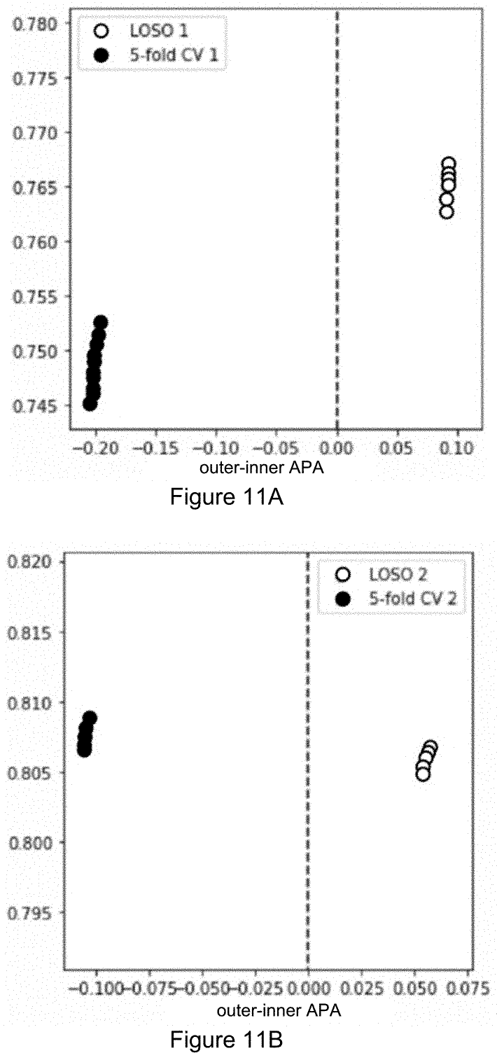

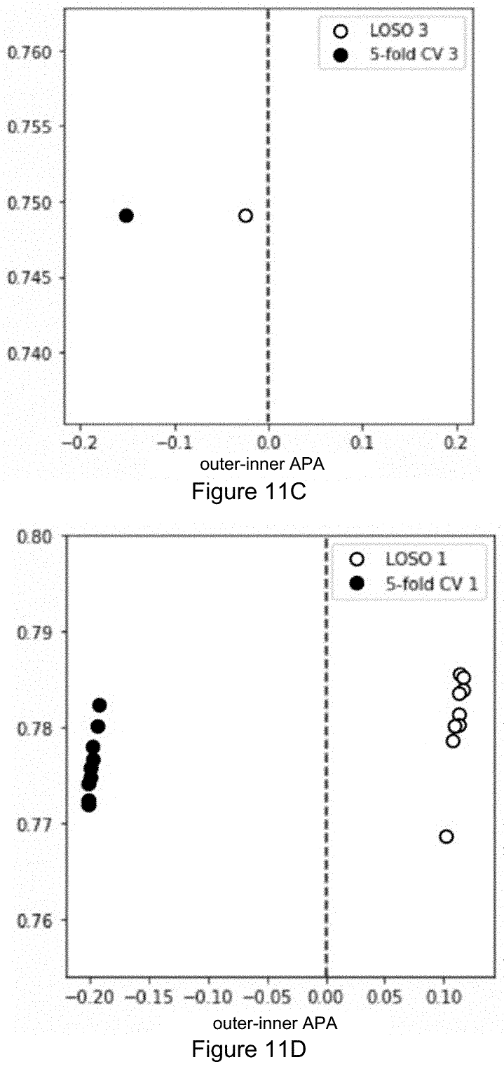

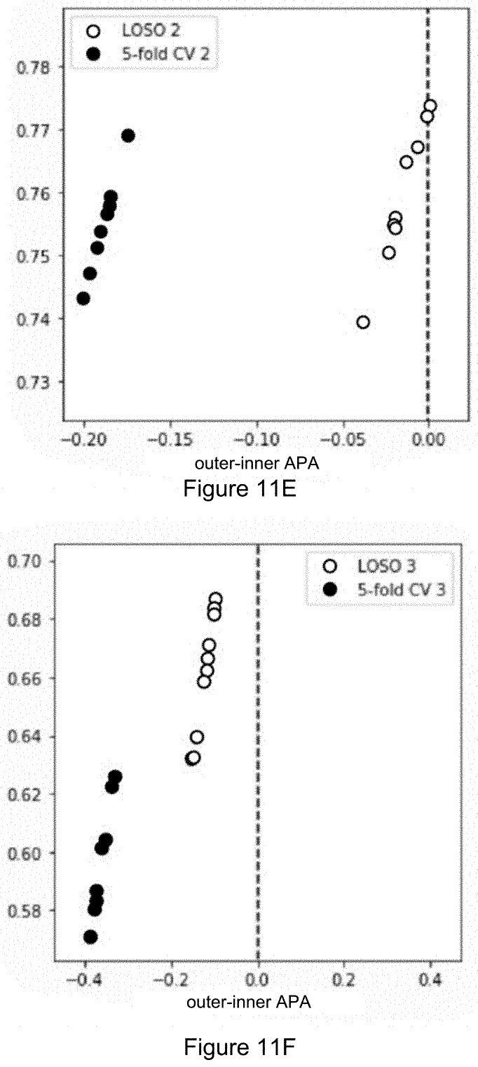

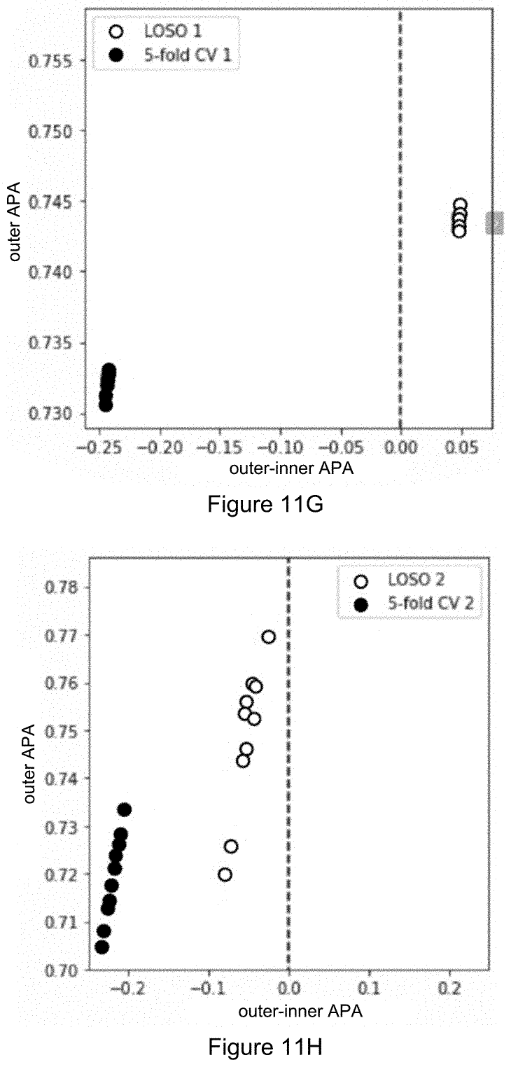

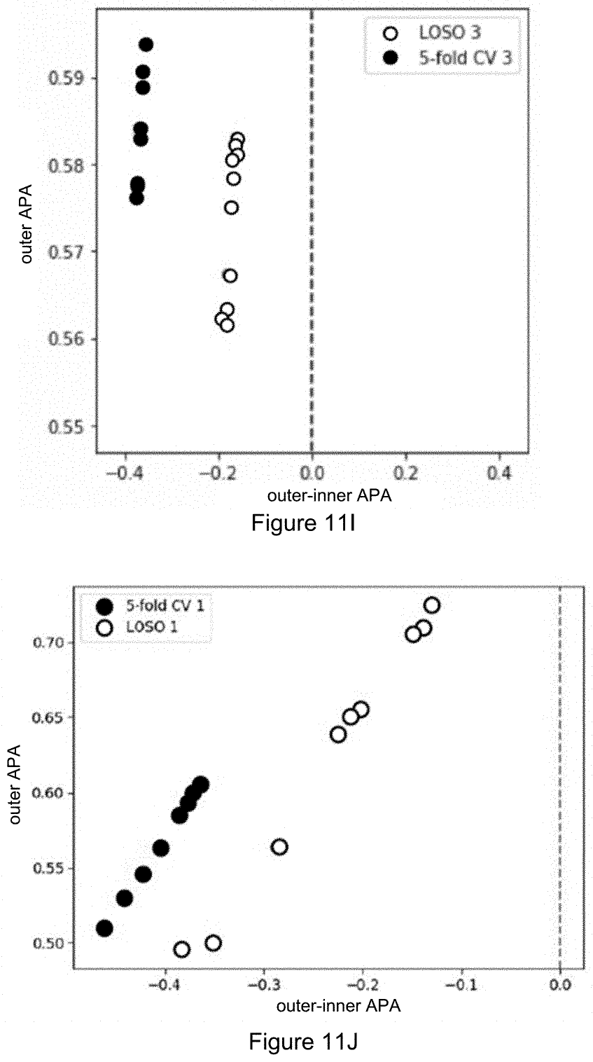

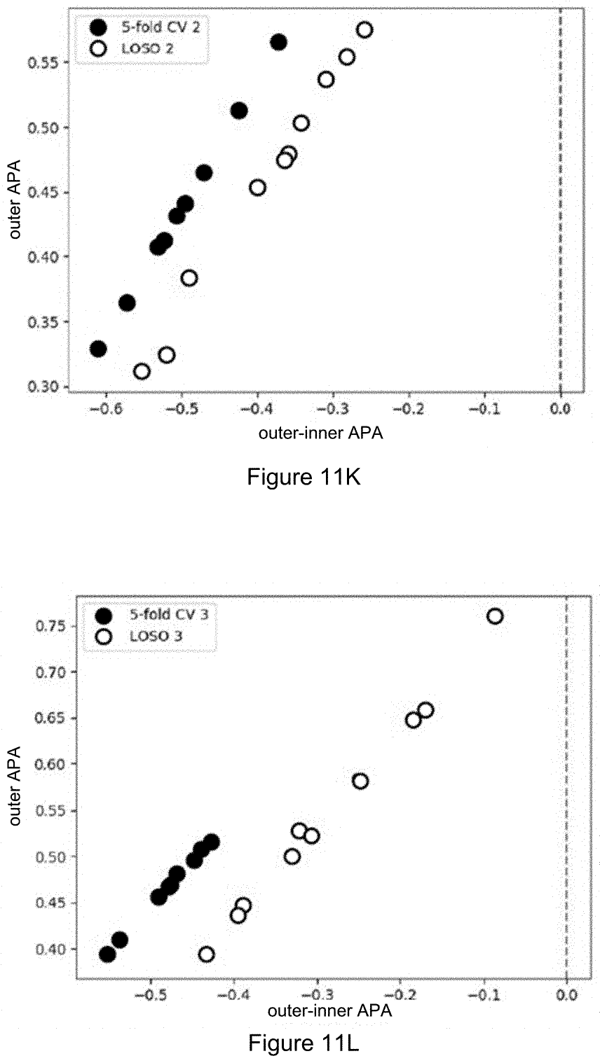

The analyses were performed using 29 normalized expression profiles as input features, and 6 GM scores as input features to the classifiers. The rationale for using the 6 GM scores was that in prior research and preliminary analyses (internal data, not shown) it showed very promising results. The results are shown in FIGS. 10A through 11L.

In all analyses, except one of the GM logistic regression runs, LOSO CV AUC estimates were closer to the test set values than k-fold CV estimates. This is demonstrated by the closeness of the black (LOSO) dots to vertical dashed line compared with the dark gray (k-fold) dots. On the basis of this finding, the rest of the analyses used LOSO.

Furthermore, the analyses showed that test set performance was superior using the 6 GM scores compared with 29-gene expression features. Table 3 shows comparison of the test set APAs for the two sets of features and different classifiers. The model selection criteria for this comparison used LOSO, because of the previous finding that LOSO has significantly lesser bias.

TABLE 3
Comparison of test set performance using GM scores and gene
expression as input features. The table contains APA values for GM scores (GMS) and 29
gene expression values (GENEX). The APA columns contain average values of the 10
models shown in FIG. 11, for the three HCV test sets. The best models were found using
LOSO cross-validation method. For each GMS/GENEX pair, the higher APA is indicated by
the bold letters.

| Classifier | GMS 1 | GENEX 1 | GMS 2 | GENEX 2 | GMS 3 | GENEX 3 |
|---|---|---|---|---|---|---|
| LR | 0.75 | 0.76 | 0.82 | 0.81 | 0.75 | 0.71 |
| SVM | 0.78 | 0.74 | 0.89 | 0.75 | 0.66 | 0.57 |
| XGBoost | 0.78 | 0.78 | 0.80 | 0.76 | 0.68 | 0.66 |
| MLP | 0.74 | 0.64 | 0.78 | 0.46 | 0.71 | 0.55 |

As seen in Table 3, GMS scores yielded higher performance in almost all cases. Based on this finding, the rest of the analyses used the GM scores as input features to classification algorithms. The use of such GM scores is an instantiation of the module 152/summarization algorithm 156 discussed above in conjunction with FIGS. 1A and 1B.

Classifier Development

To develop the classifier, a hyperparameter search was performed for the four different models. The search was performed using the LOSO cross-validation approach, and 6 GM scores as input features. For each configuration, LOSO learning was performed and predicted probabilities in the left-out datasets were pooled. The result was, for each configuration, a set of predicted probabilities for all samples in the training set. APA was then calculated using the pooled probabilities, and hyperparameter configurations were ranked using the APA values. The best configuration was the one with largest APA. Summarized LOSO results for the different algorithms are given in Table 4.

TABLE 4
LOSO training results. “APA LOSO” columns contain the LOSO-
cross-validation statistic for the best-performing hyperparameter
configuration of the corresponding model.

| Model | APA LOSO |
|---|---|
| Multi-layer Perceptron | 0.87 |
| Support Vector Machine | 0.85 |
| XGBoost | 0.77 |
| Logistic Regression | 0.76 |

Among the four classifiers, MLP gave best LOSO cross-validation APA results. The winning configuration used the following hyperparameters: two hidden layers, four nodes per hidden layer, 250 iterations, linear activation, no dropout, learning rate=1e-5, batch size=128, batch normalization, regularization: L1 (penalty=0.1), and input layer weight initialization using weight priors. Table 5 contains additional performance statistics estimated using the pooled LOSO probabilities for the winning configuration.

TABLE 5
Detailed LOSO statistics for the
winning neural network classifier.

| Statistic | Estimate |
|---|---|
| Brier score | 0.41 |
| Bacterial accuracy | 70% |
| Viral accuracy | 82% |
| Noninfected accuracy | 43% |
| Average Accuracy | 68% |
| Cross-entropy loss | 0.71 |

This analyses suggested that network performance was sensitive to the pseudo-random initialization of the network weights. To explore the space of those initial start points, additional LOSO analysis was performed for the model with the winning hyperparameter configuration, and using 5000 different random initializations of the network weights (using the weight priors, as specified by the selected configuration). The networks were trained and assessed using the same approach as in the initial run, e.g., by pooling the predicted probabilities for all folds in the LOSO run and calculating APA over the pooled probabilities. The winning seed was the one corresponding to the model with the highest APA.

The locked final model was applied to the validation clinical data. That is, the validation clinical results were computed by applying the locked classifier to the validation clinical NanoString expression data. This produced three class probabilities for each sample: bacterial, viral and non-infected. The utility of the classifier was evaluated by comparing the predictions with the clinically adjudicated diagnoses, using multiple clinically-relevant statistics. Table 6 contains the results.

TABLE 6
Performance statistics of the BVN1 classifier applied
to the independent validation clinical samples (n = 104).

| Statistic | Point estimate [95% CI] |
|---|---|
| APA | 0.83 |
| Bacterial-vs-other AUROC | 0.85 |
| Viral-vs-other AUROC | 0.88 |
| Noninfected-vs-other AUROC | 0.77 |
| Bacterial accuracy | 80% |
| Viral accuracy | 50% |
| Noninfected accuracy | 62% |

In clinical use, the key variables of interest when diagnosing a patient are expected to be the probability of bacterial and viral infections. These values are emitted by the top (softmax) layer of the neural network.

### DISCUSSION

As described above, a machine learning classifier was developed for diagnosing bacterial and viral sepsis in patients suspected of the condition, and initial validation of independent test data was performed. The project faced several major challenges. First, with respect to platform transfer, the classifier was developed using exclusively public domain data, assayed on various microarray chips. In contrast, the test data was assayed using NanoString, a platform never previously encountered in training. Second, there was significant heterogeneity between the available training datasets. Third, there was a relatively small training sample size, especially considering the problem with heterogeneity in the training data. To approach these challenges, multiple research directions were applied.

First, methods for selecting the best machine learning models for sepsis classification were investigated. The research to date indicated that due to very significant amount of technical and biological heterogeneity in the sepsis data, the standard random cross-validation produces excessive optimistic bias. Based on empirical findings, and prior research on the subject, a leave-one-study (LOSO) approach was selected for the classifier development.

Next, the impact of input feature engineering was analyzed. LOSO consistently favored custom-engineered inputs consisting of six geometric mean scores, which were therefore used as inputs to the final locked classifier. This is a somewhat unexpected result which warrants further research, including the possibility of automatically learning and improving the feature engineering transformations.

The probability distributions on the independent test data exhibited clear trends in the expected direction, in the sense that bacterial probabilities for bacterial samples tended to be high, as do viral probabilities for viral samples. Furthermore, non-infected samples had trended toward lower bacterial and viral probabilities. These trends are quantified by favorable pairwise AUROC estimates and class-conditional accuracies. Nevertheless, a significant residual overlap among the distributions is also noted, and is the focus of ongoing research.

The current attempt at platform transfer has been successful. Nevertheless, to improve the test clinical performance, future enhancements of our sepsis classifier shall add NanoString data to the training set.

This research demonstrated the feasibility of successfully learning complex sepsis classifiers using public data, and subsequently transferring to previously unseen samples assayed on previously unseen platform. To our knowledge, this has not been reported previously in the sepsis literature, and perhaps not elsewhere in molecular diagnostics.

### CONCLUSION

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A computer system for evaluating a clinical condition of a test subject of a species using an a priori grouping of features, wherein the a priori grouping of features comprises a plurality of modules, each respective module in the plurality of modules comprising an independent plurality of features whose corresponding feature values each associate with either an absence, presence or stage of an independent phenotype associated with the clinical condition, the computer system comprising:
at least one processor; and
a memory storing at least one program for execution by the at least one processor, the at least one program comprising instructions for:
(A) obtaining in electronic form a first training dataset, wherein the first training dataset comprises, for each respective training subject in a first plurality of training subjects of the species: (i) a first plurality of feature values, acquired through a first technical background using a biological sample of the respective training subject, for the independent plurality of features, in a first form that is one of transcriptomic, proteomic, or metabolomic, of at least a first module in the plurality of modules and (ii) an indication of the absence, presence or stage of a first independent phenotype corresponding to the first module, in the respective training subject;
(B) obtaining in electronic form a second training dataset, wherein the second training dataset comprises, for each respective training subject in a second plurality of training subjects of the species: (i) a second plurality of feature values, acquired through a second technical background other than the first technical background using a biological sample of the respective training subject, for the independent plurality of features, in a second form identical to the first form, of at least the first module and (ii) an indication of the absence, presence or stage of the first independent phenotype in the respective training subject;
(C) co-normalizing feature values for features present in at least the first and second training datasets across at least the first and second training datasets to remove an inter-dataset batch effect, thereby calculating, for each respective training subject in the first plurality of training subjects and for each respective training subject in the second plurality of training subjects, co-normalized feature values of at least the first module for the respective training subject; and
(D) training a main classifier, against a composite training set, to evaluate the test subject for the clinical condition, the composite training set comprising, for each respective training subject in the first plurality of training subjects and for each respective training subject in the second plurality of training subjects: (i) a summarization of the co-normalized feature values of the first module and (ii) an indication of the absence, presence or stage of the first independent phenotype in the respective training subject.

2. The computer system of claim 1, wherein each respective feature in the first module corresponds to a biomarker that associates with the first independent phenotype by being statistically significantly more abundant in subjects that exhibit the first independent phenotype as compared to subjects that do not exhibit the independent phenotype across a cohort of subjects of the species.

3. The computer system of claim 1, wherein each respective feature in the first module corresponds to a biomarker that associates with the first independent phenotype by being statistically significantly less abundant in subjects that exhibit the first independent phenotype as compared to subjects that do not exhibit the independent phenotype across a cohort of subjects of the species.

4-8. (canceled)

9. The computer system of claim 1, wherein a feature value for a first feature in a module in the plurality of modules is a linear or nonlinear combination of the feature values of each respective component in a group of components obtained by physical measurement of each respective component in the biological sample of the reference subject, wherein each respective component in the group of components is a nucleic acid, a protein, or a metabolite.

10-11. (canceled)

12. The computer system of claim 1, wherein the first form is transcriptomic, the first technical background is RNAseq, and the second technical background is a DNA microarray.

13-16. (canceled)

17. The computer system of claim 1, wherein
the first independent phenotype represents a diseased condition,
a first subset of the first training dataset consists of subjects that are free of the diseased condition,
a first subset of the second training dataset consists of subjects that are free of the diseased condition,
the co-normalizing feature values present in at least the first and second training datasets comprises estimating the inter-dataset batch effect between the first and second training dataset using only the first subset of the respective first and second training datasets.

18. The computer system of claim 17, wherein the inter-dataset batch effect includes an additive component and a multiplicative component and the co-normalizing solves an ordinary least-squares model for feature values across the first subset of the respective first and second training datasets and shrinks resulting parameters representing the additive component and a multiplicative component using an empirical Bayes estimator.

19. The computer system of claim 1, wherein the co-normalizing feature values present in at least the first and second training datasets across at least the first and second training datasets comprises estimating the inter-dataset batch effect between the first and second training datasets.

20. The computer system of claim 19, wherein the inter-dataset batch effect includes an additive component and a multiplicative component and the co-normalizing solves an ordinary least-squares model for feature values across the respective first and second training datasets and shrinks resulting parameters representing the additive component and a multiplicative component using an empirical Bayes estimator.

21. The computer system of claim 1, wherein the co-normalizing feature values present in at least the first and second training datasets across at least the first and second training datasets comprises making use of nonvariant features or quantile normalization.

22. The computer system of claim 1, wherein
each feature in the first and second dataset is a nucleic acid,
the first technical background is a first form of microarray experiment selected from the group consisting of cDNA microarray, oligonucleotide microarray, BAC microarray, and single nucleotide polymorphism (SNP) microarray,
the second technical background is a second form of microarray experiment other than first form of microarray experiment selected from the group consisting of cDNA microarray, oligonucleotide microarray, BAC microarray, and SNP microarray, and
the co-normalizing is robust multi-array average (RMA) or GeneChip robust multi-array average (GC-RMA).

23. (canceled)

24. The computer system of claim 1, wherein, for each respective training subject in the first plurality of training subjects and for each respective training subject in the second plurality of training subjects, the summarization of the co-normalized feature values of the first module is a measure of central tendency of the co-normalized feature values of the first module in the biological sample obtained from the respective training subject.

25. (canceled)

26. The computer system of claim 1, wherein, for each respective training subject in the first plurality of training subjects and for each respective training subject in the second plurality of training subjects, the summarization of the co-normalized feature values of the first module is an output of a component classifier associated with the first module upon input of the co-normalized feature values of the first module in the biological sample obtained from the respective training subject.

27. (canceled)

28. The computer system of claim 1, wherein:
the at least one program further comprises instructions for obtaining in electronic form a plurality of additional training datasets in addition to the first and second training dataset, wherein each respective additional dataset in the plurality of additional datasets comprises, for each respective training subject in an independent respective plurality of training subjects of the species: (i) a plurality of feature values, acquired through an independent respective technical background using a biological sample of the respective training subject, for an independent plurality of features, in the first form, of a respective module in the plurality of modules and (ii) an indication of the absence, presence or stage of a respective phenotype in the respective training subject corresponding to the respective module, and
the co-normalizing (C) further comprises co-normalizing feature values of features present in respective two or more training datasets in a training group comprising the first training dataset, the second training dataset and the plurality of additional training datasets, across at least the two or more respective training datasets in the training group to remove the inter-dataset batch effect, thereby calculating for each respective training subject in each respective two or more training datasets in the plurality of training datasets, co-normalized feature values of each module in the plurality of modules, and
the composite training set further comprises, for each respective training subject in each training dataset in the training group: (i) a summarization of the co-normalized feature values of a module, in the plurality of modules, in the respective training subject and (ii) an indication of the absence, presence or stage of a corresponding independent phenotype in the respective training subject.

29-31. (canceled)

32. The computer system of claim 30, wherein
the first training dataset further comprises, for each respective training subject in the first plurality of training subjects of the species: (iii) a plurality of feature values, acquired through the first technical background using the biological sample of the respective training subject of a second module in the plurality of modules and (iv) an indication of the absence, presence or stage of a second independent phenotype in the respective training subject,
the second training dataset further comprises, for each respective training subject in the second plurality of training subjects of the species: (iii) a plurality of feature values, acquired through the second technical background using the biological sample of the respective training subject of the second module and (iv) an indication of the absence, presence or stage of the second independent phenotype in the respective training subject,
the first independent phenotype and the second independent phenotype are the same as the clinical condition,
each respective feature in the first module associates with the first independent phenotype by having a feature value that is statistically significantly greater in subjects that exhibit the first independent phenotype as compared to subjects that do not exhibit the independent phenotype across a cohort of the species, and
each respective feature in the second module associates with the first independent phenotype by having a feature value that is statistically significantly fewer in subjects that exhibit the first independent phenotype as compared to subjects that do not exhibit the independent phenotype across a cohort of the species.

33-51. (canceled)

52. A computer system for evaluating a clinical condition of a test subject of a species, the computer system comprising:
at least one processor; and
a memory storing at least one program for execution by the at least one processor, the at least one program comprising instructions for:
(A) obtaining in electronic form a first training dataset, wherein the first training dataset comprises, for each respective training subject in a first plurality of training subjects of the species: (i) a first plurality of feature values, acquired using a biological sample of the respective training subject, for a plurality of features and (ii) an indication of the absence, presence or stage of a first independent phenotype in the respective training subject, wherein the first independent phenotype represents a diseased condition, and wherein a first subset of the first training dataset consists of subjects that are free of the diseased condition;
(B) obtaining in electronic form a second training dataset, wherein the second training dataset comprises, for each respective training subject in a second plurality of training subjects of the species: (i) a second plurality of feature values, acquired using a biological sample of the respective training subject, for the plurality of features and (ii) an indication of the absence, presence or stage of the first independent phenotype in the respective training subject and wherein a first subset of the second training dataset consists of subjects that are free of the diseased condition;
(C) co-normalizing feature values for a subset of the plurality of features across at least the first and second training datasets to remove an inter-dataset batch effect, wherein
the subset of features is present in at least the first and second training datasets,
the co-normalizing comprises estimating an inter-dataset batch effect between the first and second training dataset using only the first subset of the respective first and second training datasets, and
the inter-dataset batch effect includes an additive component and a multiplicative component and the co-normalizing solves an ordinary least-squares model for feature values across the first subset of the respective first and second training datasets and shrinks resulting parameters representing the additive component and a multiplicative component using an empirical Bayes estimator, thereby calculating using the resulting parameters: for each respective training subject in the first plurality of training subjects and for each respective training subject in the second plurality of training subjects, co-normalized feature values of the subset of the plurality of features; and
(D) training a main classifier, against a composite training set, to evaluate the test subject for the clinical condition, the composite training set comprising: for each respective training subject in the first plurality of training subjects and for each respective training subject in the second plurality of training subjects: (i) co-normalized feature values of the subset of the plurality of features and (ii) the indication of the absence, presence or stage of the first independent phenotype in the respective training subject.

53-76. (canceled)

77. A computer system for dataset co-normalization, the computer system comprising:
at least one processor; and
a memory storing at least one program for execution by the at least one processor, the at least one program comprising instructions for:
(A) obtaining in electronic form a first training dataset, wherein the first training dataset comprises, for each respective training subject in a first plurality of training subjects of the species: (i) a first plurality of feature values, acquired using a biological sample of the respective training subject, for a plurality of features and (ii) an indication of the absence, presence or stage of a clinical condition in the respective training subject, and wherein a first subset of the first training dataset consists of subjects do not exhibit the clinical condition;
(B) obtaining in electronic form a second training dataset, wherein the second training dataset comprises, for each respective training subject in a second plurality of training subjects of the species: (i) a second plurality of feature values, acquired using a biological sample of the respective training subject, for the plurality of features and (ii) an indication of the absence, presence or stage of the clinical condition in the respective training subject and wherein a first subset of the second training dataset consists of subjects that do not exhibit the clinical condition;
(C) estimating an initial mean absolute deviation between (i) a vector of average expression of the subset of the plurality of features across the first plurality of subjects and (ii) a vector of average expression of the subset of the plurality of features across the second plurality of subjects;
(D) co-normalizing feature values for a subset of the plurality of features across at least the first and second training datasets to remove an inter-dataset batch effect, wherein
the subset of features is present in at least the first and second training datasets,
the co-normalizing comprises estimating an inter-dataset batch effect between the first and second training dataset using only the first subset of the respective first and second training datasets, and
the inter-dataset batch effect includes an additive component and a multiplicative component and the co-normalizing solves an ordinary least-squares model for feature values across the first subset of the respective first and second training datasets and shrinks resulting parameters representing the additive component and the multiplicative component using an empirical Bayes estimator, thereby calculating using the resulting parameters: for each respective training subject in the first subset of training subjects, co-normalized feature values of each feature value in the plurality of features;
(E) estimating a post co-normalization mean absolute deviation between (i) a vector of average expression of the co-normalized feature values of the plurality of features across the first training dataset and (ii) a vector of average expression of the subset of the plurality of features across the second training dataset; and
(F) repeating the co-normalizing (D) and the estimating (E) until the co-normalization mean absolute deviation converges.

78-87. (canceled)

88. A computer system for evaluating a clinical condition of a test subject of a species using an a priori grouping of features, wherein the a priori grouping of features comprises a plurality of modules, each respective module in the plurality of modules comprising an independent plurality of features whose corresponding feature values each associate with either an absence, presence or stage of the clinical condition, the computer system comprising:
at least one processor; and
a memory storing at least one program for execution by the at least one processor, the at least one program comprising instructions for:
(A) obtaining in electronic form a first training dataset, wherein the first training dataset comprises, for each respective training subject in a first plurality of training subjects of the species: (i) a first plurality of feature values, acquired through a first technical background using a biological sample of the respective training subject, for the independent plurality of features, in a first form that is one of transcriptomic, proteomic, or metabolomic, of at least a first module in the plurality of modules and (ii) an indication of the absence, presence or stage of the clinical condition, in the respective training subject;
(B) obtaining in electronic form a second training dataset, wherein the second training dataset comprises, for each respective training subject in a second plurality of training subjects of the species: (i) a second plurality of feature values, acquired through a second technical background other than the first technical background using a biological sample of the respective training subject, for the independent plurality of features, in a second form identical to the first form, of at least the first module and (ii) an indication of the absence, presence or stage of the clinical condition in the respective training subject;
(C) co-normalizing feature values for features present in at least the first and second training datasets across at least the first and second training datasets to remove an inter-dataset batch effect, thereby calculating, for each respective training subject in the first plurality of training subjects and for each respective training subject in the second plurality of training subjects, co-normalized feature values of at least the first module for the respective training subject; and
(D) training a main classifier, against a composite training set, to evaluate the test subject for the clinical condition, the composite training set comprising, for each respective training subject in the first plurality of training subjects and for each respective training subject in the second plurality of training subjects: (i) a summarization of the co-normalized feature values of the first module and (ii) an indication of the absence, presence or stage of the clinical condition in the respective training subject.

89. A computer system for evaluating a clinical condition of a test subject of a species using a grouping of features, wherein the grouping of features comprises a plurality of modules, each respective module in the plurality of modules comprising an independent plurality of features whose corresponding feature values each associate with either an absence, presence or stage of a phenotype associated with the clinical condition, the computer system comprising:
at least one processor; and
a memory storing at least one program for execution by the at least one processor, the at least one program comprising instructions for:
(A) obtaining in electronic form a first training dataset, wherein the first training dataset comprises, for each respective training subject in a first plurality of training subjects of the species: (i) for each respective module in the plurality of modules, a plurality of feature values for the independent plurality of features obtained from a biological sample from the respective training subject, and (ii) an indication of the absence, presence or stage of the clinical condition in the respective training subject;
(B) summarizing, for each respective training subject in the first plurality of training subjects, for each respective module in the plurality of modules, the plurality of feature values, thereby forming a corresponding summarization of the feature values of the respective module for each respective training subject; and
(C) training a main classifier, against a composite training set, to evaluate the test subject for the clinical condition, the composite training set comprising, for each respective training subject in the first plurality of training subjects: (i) for each respective module in the plurality of modules, the corresponding summarization of the feature values of the respective module and (ii) the indication of the absence, presence or stage of the clinical condition in the respective training subject.

90-113. (canceled)

114. A computer system for evaluating a clinical condition of a test subject of a species using a grouping of features, wherein the grouping of features comprises a plurality of modules, each respective module in the plurality of modules comprising an independent plurality of features whose corresponding feature values each associate with either an absence, presence or stage of a phenotype associated with the clinical condition, the computer system comprising:
at least one processor; and
a memory storing at least one program for execution by the at least one processor, the at least one program comprising instructions for:
(A) obtaining in electronic form a test dataset, wherein the test dataset comprises, for each respective module in the plurality of modules, a plurality of feature values for the independent plurality of features obtained from a biological sample from the test subject;
(B) summarizing, for each respective module in the plurality of modules, the plurality of feature values, thereby forming a corresponding summarization of the feature values of the respective module for the test subject; and
(C) inputting, for each respective module in the plurality of modules, the corresponding summarization of the feature values of the respective module into a classifier trained to distinguish between two or more classes of the clinical condition, thereby providing a classification of the clinical condition for the test subject.

115. (canceled)
